(19) United States
(12) Patent Application Publication
(10) Pub. No.: US 2026/0244096 A1
(43) Pub. Date: Aug. 20, 2026

(54) THERMAL ACID GENERATORS, COATING COMPOSITIONS, AND PATTERN FORMATION METHODS

(71) Applicant: DuPont Electronic Materials International, LLC, Marlborough, MA (US)

(72) Inventors: Jung June Lee, Hwaseong-si (KR); Paul J. LaBeaume, Auburn, MA (US); Emad Aqad, Northborough, MA (US); Tomas Marangoni, Orono, ME (US); Mingqi Li, Shrewsbury, MA (US); James F. Cameron, Brookline, MA (US); Edon Vitaku, Marlborough, MA (US); Kenneth Hernandez, Ashland, MA (US)

(21) Appl. No.: 19/529,660

(22) Filed: Feb. 4, 2026

Related U.S. Application Data
(60) Provisional application No. 63/758,678, filed on Feb. 14, 2025.

(51) Int. Cl. G03F 7/004; C07C 211/63; C07C 211/64; C07C 309/60; C07D 213/20; G03F 7/039; G03F 7/095; G03F 7/16

(52) U.S. Cl. CPC G03F 7/0045; C07C 211/63; C07C 211/64; C07C 309/60; C07D 213/20; G03F 7/0392; G03F 7/095; G03F 7/168

(57) ABSTRACT

A thermal acid generator compound comprising an anion and a nitrogen-containing cation, wherein the anion is represented by Formula (1):

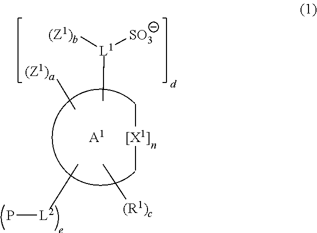

wherein, in Formula (1), ring A¹ is a monocyclic C₃₋₆₀ aromatic group, a polycyclic C₃₋₆₀ aromatic group, a C₃₋₁₅ monoalicyclic group, or a C₃₋₁₅ polyalicyclic group, each L¹ is independently a single bond or one or more linking groups, wherein L¹ is free of fluorine, and each of the remaining groups are as defined herein.

### CROSS-REFERENCE TO RELATED APPLICATIONS

This application claims priority to and the benefit of U.S. Provisional Application Ser. No. 63/758,678, filed on Feb. 14, 2025, in the United States Patent Trademark Office, the entire contents of which are incorporated by reference herein.

### FIELD

The present invention relates to thermal acid generators, coating compositions, and to pattern formation methods using such coating compositions. The invention finds particular applicability in lithographic applications in the semiconductor manufacturing industry.

### BACKGROUND

Photoresist underlayer compositions are used in the semiconductor industry as etch masks for lithography in advanced technology nodes for integrated circuit manufacturing. These compositions are often used in tri-layer and quad-layer photoresist integration schemes, where an organic or silicon containing anti-reflective coating and a patternable photoresist film layers are disposed on the bottom layer having a high carbon content.

When a chemically amplified photoresist composition for ArF or extreme ultraviolet (EUV) lithography is prepared using a resin that may include by-products, the number of defects (surface defects) in the resist pattern following developing can become problematic, even if properties as the sensitivity, the resolution, and the resist pattern shape are satisfactory. These surface defects refer to problems such as scum and bridging between resist patterns.

EUV lithography is a leading technology to replace optical lithography for volume semiconductor manufacturing at feature sizes of several nanometers. Currently, EUV lithography has become the preferred patterning technology over 193 nm immersion processes for high volume manufacturing for product nodes under 10 nm. In EUV lithography, there are fewer photons at the exposed areas than in the exposed areas of ArF lithography. Due to the lack of photons and shrunken pitches of patterns, impact of shot noise becomes more significant on pattern profiles. Scum and bridging defects between resist patterns, such as a nano-bridging defect at line to space patterns with 3×nm pitch, may cause fatal bridging defects after whole pattern transfer by an etching process.

Accordingly, there remains a need for new photoresist underlayer materials that can reduce the extent of film stripping loss and/or mitigate scum and bridge defects in photoresists for ArF and EUV lithography.

### SUMMARY

An aspect is directed to a thermal acid generator compound including an anion and a nitrogen-containing cation, wherein the anion is represented by Formula (1):

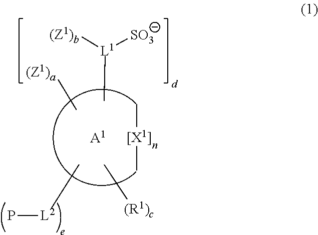

wherein, in Formula (1), ring A¹ is a monocyclic C₃₋₆₀ aromatic group, a polycyclic C₃₋₆₀ aromatic group, a C₃₋₁₅ monoalicyclic group, or a C₃₋₁₅ polyalicyclic group, each L¹ is independently a single bond or one or more linking groups, wherein L¹ is free of fluorine, each L² is independently a single bond or one or more divalent linking groups, P is a polymerizable group, each R¹ is independently a monovalent non-hydrogen substituent; wherein each R¹ optionally further comprises one or more divalent linking groups as part of its structure, each X¹ is independently —O—, —S—, —N(R^{2a})—, —C(O)—, —S(O)R^{2a}—, or —S(O₂)R^{2a}—, wherein R^{2a} is independently chosen from substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, each Z¹ independently comprises an anion stabilizing group, wherein at least one Z¹ is configured to form an intramolecular non-covalent bond with the sulfonate anion group to form a ring having from 5 to 9 ring atoms, or from 5 to 8 ring atoms, wherein Z¹ is independently chosen from —OH, —C(O)OH, —SH, —C(O)SH, —NHS(O)₂R³, —S(O)₂R³, —S(O)R³, —S(O)₂NHS(O)₂R³, —CH(═NOH), or —B(R⁴)₂; wherein each Z¹ optionally further comprises one or more divalent linking groups as part of its structure, each R³ is independently chosen from trifluoromethyl, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, each R⁴ is independently chosen from hydrogen, fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, two R¹ together optionally form a fused ring with Cy¹, wherein the fused ring optionally further comprises one or more divalent linking groups as part of its structure, one Z¹ and one R¹ together optionally form a fused ring with Cy¹, wherein the fused ring optionally further comprises one or more divalent linking groups as part of its structure, each a and b is independently an integer from 0 to 2, provided that a sum of a and b is 1 or greater, c is an integer from 0 to 10, d is an integer from 1 to 3, e is 0 or 1, and n is an integer from 0 to 4.

Another aspect is directed to a coating composition that includes the thermal acid generator, a polymer, and a solvent.

Still another aspect is directed to a patterning method including applying a layer of the coating composition of claim 10 on a substrate to provide an underlayer composition; curing the underlayer composition to form an underlayer film; applying a layer of a photoresist composition on the underlayer film to provide a photoresist composition layer; pattern-wise exposing the photoresist composition layer to activating radiation to provide an exposed photoresist composition layer; and developing the exposed photoresist composition layer to provide a resist relief image.

### DETAILED DESCRIPTION

Reference will now be made in detail to exemplary embodiments, examples of which are illustrated in the present description. In this regard, the present exemplary embodiments may have different forms and should not be construed as being limited to the descriptions set forth herein. Accordingly, the exemplary embodiments are merely described below to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “a,” “an,” and “the” do not denote a limitation of quantity and are to be construed to cover both the singular and the plural, unless otherwise indicated herein or clearly contradicted by context. “Or” means “and/or” unless clearly indicated otherwise. The modifier “about” used in connection with a quantity is inclusive of the stated value and has the meaning dictated by the context (e.g., includes the degree of error associated with measurement of the particular quantity). All ranges disclosed herein are inclusive of the endpoints, and the endpoints are independently combinable with each other. The suffix “(s)” is intended to include both the singular and the plural of the term that it modifies, thereby including at least one of that term. “Optional” or “optionally” means that the subsequently described event or circumstance can or cannot occur, and that the description includes instances where the event occurs and instances where it does not. The terms “first,” “second,” and the like, herein do not denote an order, quantity, or importance, but rather are used to distinguish one element from another. When an element is referred to as being “on” another element, it may be directly in contact with the other element or intervening elements may be present therebetween. In contrast, when an element is referred to as being “directly on” another element, there are no intervening elements present. It is to be understood that the described components, elements, limitations, and/or features of aspects may be combined in any suitable manner in the various aspects.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “actinic rays” or “radiation” means, for example, a bright line spectrum of a mercury lamp, far ultraviolet rays represented by an excimer laser, extreme ultraviolet rays (EUV light), X-rays, particle rays such as electron beams and ion beams, or the like. In addition, in the present invention, “light” means actinic rays or radiation. The krypton fluoride laser (KrF laser) is a particular type of excimer laser, which is sometimes referred to as an exciplex laser. “Excimer” is short for “excited dimer,” while “exciplex” is short for “excited complex.” An excimer laser uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range. Furthermore, “exposure” in the present specification includes, unless otherwise specified, not only exposure by a mercury lamp, far ultraviolet rays represented by an excimer laser, X-rays, extreme ultraviolet rays (EUV light), or the like, but also writing by particle rays such as electron beams and ion beams.

As used herein, the term “hydrocarbon” refers to an organic compound or group having at least one carbon atom and at least one hydrogen atom; “alkyl” refers to a straight or branched chain saturated hydrocarbon group having the specified number of carbon atoms and having a valence of one; “alkylene” refers to an alkyl group having a valence of two; “hydroxyalkyl” refers to an alkyl group substituted with at least one hydroxyl group (—OH); “alkoxy” refers to “alkyl-O—”; “carboxyl” and “carboxylic acid group” refer to a group having the formula “—C(O)—OH”; “cycloalkyl” refers to a monovalent group having one or more saturated rings in which all ring members are carbon; “cycloalkylene” refers to a cycloalkyl group having a valence of two; “alkenyl” refers to a straight or branched chain, monovalent hydrocarbon group having at least one carbon-carbon double bond; “alkenoxy” refers to “alkenyl-O—”; “alkenylene” refers to an alkenyl group having a valence of two; “cycloalkenyl” refers to a non-aromatic cyclic divalent hydrocarbon group having at least three carbon atoms, with at least one carbon-carbon double bond; “alkynyl” refers to a monovalent hydrocarbon group having at least one carbon-carbon triple bond; the term “aromatic group” refers to a monocyclic or polycyclic aromatic ring system that satisfies Huckel's Rule (4n+2π electrons) and includes carbon atoms in the ring; the term “heteroaromatic group” refers to an aromatic group that includes one or more heteroatoms (e.g., 1-4 heteroatoms) selected from N, O, and S instead of a carbon atom in the ring; “aryl” refers to a monovalent monocyclic or polycyclic aromatic ring system where every ring member is carbon, and may include a group with an aromatic ring fused to at least one cycloalkyl or heterocycloalkyl ring; “arylene” refers to an aryl group having a valence of two; “alkylaryl” refers to an aryl group that has been substituted with an alkyl group; “arylalkyl” refers to an alkyl group that has been substituted with an aryl group; “aryloxy” refers to “aryl-O—”; and “arylthio” refers to “aryl-S—”.

The prefix “hetero” means that the compound or group includes at least one member that is a heteroatom (e.g., 1, 2, 3, or 4 or more heteroatom(s)) instead of a carbon atom, wherein the heteroatom(s) is each independently N, O, S, Si, or P; “heteroatom-containing group” refers to a substituent group that includes at least one heteroatom; “heteroalkyl” refers to an alkyl group having at least one heteroatom instead of carbon; “heterocycloalkyl” refers to a cycloalkyl group having 1-4 heteroatoms as ring members instead of carbon; “heterocycloalkylene” refers to a heterocycloalkyl group having a valence of two; “heteroaryl” refers to an aromatic 4-8 membered monocyclic, 8-12 membered bicyclic, or 11-14 membered tricyclic ring system having 1-4 heteroatoms (if monocyclic), 1-6 heteroatoms (if bicyclic), or 1-9 heteroatoms (if tricyclic) that are each independently selected from N, O, S, Si, or P (e.g., carbon atoms and 1-3, 1-6, or 1-9 heteroatoms of N, O, or S, if monocyclic, bicyclic, or tricyclic, respectively). Examples of heteroaryl groups include pyridyl, furyl (furyl or furanyl), imidazolyl, benzimidazolyl, pyrimidinyl, thiophenyl or thienyl, quinolinyl, indolyl, thiazolyl, and the like; and “heteroarylene” refers to a heteroaryl group having a valence of two.

The term “halogen” means a monovalent substituent that is fluorine (fluoro), chlorine (chloro), bromine (bromo), or iodine (iodo). The prefix “halo” means a group including one or more of a fluoro, chloro, bromo, or iodo substituent instead of a hydrogen atom. A combination of halo groups (e.g., bromo and fluoro), or only fluoro groups may be present. For example, the term “haloalkyl” refers to an alkyl group substituted with one or more halogens. As used herein, “substituted C₁₋₈ haloalkyl” refers to a C₁₋₈ alkyl group substituted with at least one halogen, and is further substituted with one or more other substituent groups that are not halogens. It is to be understood that substitution of a group with a halogen atom is not to be considered a heteroatom-containing group, because a halogen atom does not replace a carbon atom.

Each of the foregoing substituent groups optionally may be substituted unless expressly provided otherwise. The term “optionally substituted” refers to being substituted or unsubstituted. “Substituted” means that at least one hydrogen atom of the chemical structure or group is replaced with another terminal substituent group that is typically monovalent, provided that the designated atom's normal valence is not exceeded. When the substituent is oxo (i.e., O), then two geminal hydrogen atoms on the carbon atom are replaced with the terminal oxo group. It is further noted that the oxo group is bonded to carbon via a double bond to form a carbonyl (C═O), where the carbonyl group is represented herein as —C(O)—. Combinations of substituents or variables are permissible. Exemplary substituent groups that may be present on a “substituted” position include, but are not limited to, nitro (—NO₂), cyano (—CN), hydroxyl (—OH), oxo (O), amino (—NH₂), mono- or di-(C₁₋₆)alkylamino, alkanoyl (such as a C₂₋₆ alkanoyl group such as acyl), formyl (—C(O)H), carboxylic acid or an alkali metal or ammonium salt thereof, esters (including acrylates, methacrylates, and lactones) such as C₂₋₆ alkyl esters (—C(O)O-alkyl or —OC(O)-alkyl) and C₇₋₁₃ aryl esters (—C(O)O-aryl or —OC(O)-aryl); amido (—C(O)NR₂ wherein R is hydrogen or C₁₋₆ alkyl), carboxamido (—CH₂C(O)NR₂ wherein R is hydrogen or C₁₋₆ alkyl), halogen, thiol (—SH), C₁₋₆ alkylthio (—S-alkyl), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₂₋₁₈ heterocycloalkenyl, C₆₋₁₂ aryl having at least one aromatic ring (e.g., phenyl, biphenyl, naphthyl, or the like, each ring either substituted or unsubstituted aromatic), C₇₋₁₉ arylalkyl having 1 to 3 separate or fused rings and from 6 to 18 ring carbon atoms, arylalkoxy having 1 to 3 separate or fused rings and from 6 to 18 ring carbon atoms, C₇₋₁₂ alkylaryl, C₃₋₁₂ heterocycloalkyl, C₃₋₁₂ heteroaryl, C₁₋₆ alkyl sulfonyl (—S(O)₂-alkyl), C₆₋₁₂ arylsulfonyl (—S(O)₂-aryl), or tosyl (CH₃C₆H₄SO₂—).

As used herein, when a definition is not otherwise provided, a “divalent linking group” refers to a divalent group including one or more of —O—, —S—, —Te—, —Se—, —C(O)—, —C(O)O—, —N(R′)—, —C(O)N(R′)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀alkyl, substituted or unsubstituted C₁₋₂₀ heteroalkyl, substituted or unsubstituted C₆₋₃₀ aryl, or substituted or unsubstituted C₃₋₃₀ heteroaryl. Typically, the divalent linking group includes one or more of —O—, —S—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. More typically, the divalent linking group includes at least one of —O—, —C(O)—, —C(O)O—, —N(R′)—, —C(O)N(R′)—, substituted or unsubstituted C₁₋₁₀alkylene, substituted or unsubstituted C₃₋₁₀cycloalkylene, substituted or unsubstituted C₃₋₁₀ heterocycloalkylene, substituted or unsubstituted C₆₋₁₀ arylene, substituted or unsubstituted C₃₋₁₀ heteroarylene, or a combination thereof, wherein R is hydrogen, substituted or unsubstituted C₁₋₁₀ alkyl, substituted or unsubstituted C₁₋₁₀ heteroalkyl, substituted or unsubstituted C₆₋₁₀ aryl, or substituted or unsubstituted C₃₋₁₀ heteroaryl.

As used herein, an “acid-labile group” refers to a group in which a bond is cleaved by the action of an acid, optionally and typically with thermal treatment, resulting in formation of a polar group, such as a carboxylic acid or alcohol group. In some instances, the acid-labile group may be formed on a polymer, and optionally and typically with a moiety connected to the cleaved bond becoming disconnected from the polymer. In other systems, a non-polymeric compound may include an acid-labile group that may be cleaved by the action of an acid, resulting in formation of a polar group, such as a carboxylic acid or alcohol group on a cleaved portion of the non-polymeric compound. Such acid is typically a photo-generated acid with bond cleavage occurring during post-exposure baking (PEB); however, embodiments are not limited thereto, and, for example, such acid may be thermally generated. Suitable acid-labile groups include, for example: tertiary alkyl ester groups, secondary or tertiary ester groups having aryl groups, secondary or tertiary ester groups having a combination of alkyl and aryl groups, tertiary alkoxy groups, acetal groups, or ketal groups. Acid-labile groups are also commonly referred to in the art as “acid-cleavable groups,” “acid-cleavable protecting groups,” “acid-labile protecting groups,” “acid-leaving groups,” “acid-decomposable groups,” and “acid-sensitive groups.”

The present inventors have discovered thermal acid generators (TAGs) that include an anionic core that includes cyclic group substituted with (i) an anion group, and (ii) an anion-stabilizing group that is configured to stabilize the anion group with an intramolecular non-covalent bond. In other words, the anion stabilizing group is configured to form an intramolecular non-covalent bond with the anion group. For example, without wishing to be bound to theory, the anion stabilizing group is capable of forming an intramolecular non-covalent bond with the anion group, or, for example, the anion-stabilizing group may form an intramolecular non-covalent bond with the anion group. In some embodiments, the intramolecular non-covalent bond may be formed in-situ, such as when the thermal acid generator compound is included in a photoresist underlayer coating composition.

Provided is a thermal acid generator compound including an anion and a nitrogen-containing cation, wherein the anion is represented by Formula (1):

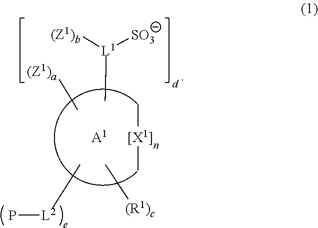

In Formula (1), ring A¹ is a monocyclic C₃₋₆₀ aromatic group, a polycyclic C₃₋₆₀ aromatic group, a C₃₋₁₅ monoalicyclic group, or a C₃₋₁₅ polyalicyclic group. For example, in some embodiments, ring A¹ may be a monocyclic C₃₋₆₀ aromatic group or a polycyclic C₃₋₆₀ aromatic group. In other embodiments, ring A¹ may be a C₃₋₁₅ monoalicyclic group or a C₃₋₁₅ polyalicyclic group. Ring A¹ optionally includes a heteroatom designated as X¹, as described herein.

In some embodiments, ring A¹ may be a monocyclic C₃₋₆₀ aromatic group or a polycyclic C₆₋₆₀ aromatic group. The term “aromatic group” includes both aromatic and heteroaromatic groups. In an embodiment, the monocyclic C₃₋₆₀ aromatic group may be a monocyclic C₃₋₆₀ arylene group or a monocyclic C₃₋₆₀ heteroarylene group, typically a monocyclic C₆₋₃₀ arylene group or a monocyclic C₃₋₃₀ heteroarylene group. It is to be understood that when the “C₃₋₆₀ aromatic group” is polycyclic, the number of carbon atoms is sufficient for the group to be chemically feasible. For example, the “polycyclic C₃₋₆₀ aromatic group” may refer to a polycyclic C₁₋₆₀ arylene group or a polycyclic C₁₀₋₃₀ arylene group. Exemplary monocyclic or polycyclic C₃₋₆₀ aromatic groups include, but are not limited to, benzene, naphthalene, anthracene, phenanthrene, pyrene, coronene, triphenylene, chrysene, phenalene, benz[a]anthracene, dibenz[a,h]anthracene, benzo[a]pyrene, or the like.

In some embodiments, ring A¹ may be a C₃₋₁₅ monoalicyclic group or a C₃₋₁₅ polyalicyclic group. For example, ring A¹ may be a C₃₋₁₅ monoalicyclic group or a C₆₋₁₅ polyalicyclic group. It is to be understood that the alicyclic group may optionally include a heteroatom group as designated as X¹ in the structure of Formula 1. For example, ring A¹ may be a monocyclic C₃₋₁₅ cycloalkylene, a monocyclic C₃₋₁₅ cycloalkenylene, a monocyclic C₃₋₁₅ heterocycloalkylene, a monocyclic C₃₋₁₅ heterocycloalkenylene, a polycyclic C₆₋₁₅ cycloalkylene, a polycyclic C₆₋₁₅ cycloalkenylene, a polycyclic C₆₋₁₅ heterocycloalkylene, or a polycyclic C₆₋₁₅ heterocycloalkenylene. Exemplary alicyclic groups for ring A¹ include, but are not limited to, cyclopentane, cyclohexane, decalin, tetrahydrofuran, thiolane, thiane, thiane-1,1,-dioxide, thiane-1-oxide, tetrahydropyran, adamantyl, bicyclo[3.2.1]octane, bicyclo[4.3.0]nonane, bicyclo[3.3.1]nonane, or the like.

In Formula (1), each L¹ is independently a single bond or one or more linking groups, wherein L¹ is free of fluorine. In other words, when L¹ is one or more linking groups, then L¹ is free of fluorine. Each of the one or more divalent linking groups may be substituted or unsubstituted. Exemplary divalent linking groups may each independently be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀alkyl, substituted or unsubstituted C₁₋₂₀ heteroalkyl, substituted or unsubstituted C₆₋₃₀ aryl, or substituted or unsubstituted C₃₋₃₀ heteroaryl. Typically, L¹ may be a single bond or substituted or unsubstituted C₁₋₂₀ alkylene, preferably a single bond or substituted or unsubstituted C₁₋₁₀ alkylene.

It is to be understood that when b is 0, then L¹ may be a single bond or one or more divalent linking groups, wherein L¹ is free of fluorine.

In Formula (1), each L² is independently a single bond or one or more divalent linking groups. Each of the one or more divalent linking groups may be substituted or unsubstituted. Exemplary divalent linking groups may each independently be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀alkyl, substituted or unsubstituted C₁₋₂₀ heteroalkyl, substituted or unsubstituted C₆₋₃₀ aryl, or substituted or unsubstituted C₃₋₃₀ heteroaryl. In some embodiments, L² comprises one or more divalent linking groups selected from —O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (1), P is a polymerizable group. Exemplary polymerizable groups include, but are not limited to, a (meth)acrylate group, a vinyl group, a vinyl aromatic group, a vinyl ether group, a vinyl ketone group, a vinyl ester group, an epoxy group, or the like, or a combination thereof.

In Formula (1), each R¹ is independently a monovalent non-hydrogen substituent; wherein each R¹ optionally further comprises one or more divalent linking groups as part of its structure. For example, each R¹ may independently be halogen, hydroxyl, substituted or unsubstituted C₁₋₃₀ alkyl, substituted or unsubstituted C₃₋₃₀ cycloalkyl, substituted or unsubstituted C₃₋₃₀cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy. Typically, each R¹ may independently be hydroxyl, substituted or unsubstituted C₁₋₃₀ alkyl, substituted or unsubstituted C₃₋₃₀cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy.

In Formula (1), each R¹ optionally further includes one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In some embodiments, one or more R¹ may each independently further include an acid-labile group, a lactone-containing group, a base-solubilizing group, or the like, or a combination thereof.

In some embodiments, one or more R¹ may each independently include polymerizable moiety as all or part of its structure. For example, R¹ may further include a (meth)acrylate group, a vinyl aromatic group, a vinyl ether group, a vinyl ketone group, and/or a vinyl ester group as all or part of its structure.

In Formula (1), each X¹ is independently —O—, —S—, —N(R^{2a})—, —C(O)—, —S(O)R^{2a}—, or —S(O₂)R^{2a}—, wherein R^{2a} is independently chosen from substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. It is to be understood that when n is 0, then X¹ is not present in the ring A¹.

In Formula (1), each Z¹ independently comprises an anion stabilizing group, wherein at least one Z¹ is configured to form an intramolecular non-covalent bond with the sulfonate anion group to form a ring having from 5 to 9 ring atoms, or from 5 to 8 ring atoms, wherein Z¹ is independently chosen from —OH, —C(O)OH, —SH, —C(O)SH, —NHS(O)₂R³, —S(O)₂R³, —S(O)R³, —S(O)₂NHS(O)₂R³, —CH(═NOH), or —B(R⁴)₂; wherein each Z¹ optionally further comprises one or more divalent linking groups as part of its structure. Typically, each anion stabilizing group Z¹ may be independently chosen from —OH, C(O)OH, SH, or —B(OH)₂, and preferably at least one anion stabilizing group Z¹ comprises —OH.

In Formula (1), each R³ is independently chosen from trifluoromethyl, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (1), each R⁴ is independently chosen from hydrogen, fluorine, hydroxyl, substituted or unsubstituted C₁₋₂₀alkyl, substituted or unsubstituted C₁₋₂₀ heteroalkyl, substituted or unsubstituted C₆₋₃₀ aryl, or substituted or unsubstituted C₃₋₃₀ heteroaryl.

For example, each Z¹ may be configured to form an intramolecular non-covalent bond with the sulfonate anion group to form a ring having from 5 to 9 ring atoms, or from 5 to 8 ring atoms, or typically 6 or 7 ring atoms. It is to be understood that when ring A¹ includes two or more anion stabilizing groups Z¹, then at least one of the anion stabilizing groups Z¹ is configured to form an intramolecular non-covalent bond with at least one sulfonate anion group to form a ring having from 5 to 9 ring atoms, or from 5 to 8 ring atoms. Similarly, when L¹ includes two or more anion stabilizing groups Z¹, then at least one of the anion stabilizing groups Z¹ is configured to form an intramolecular non-covalent bond with at least one sulfonate anion group to form a ring having from 5 to 9 ring atoms, or from 5 to 8 ring atoms.

As used herein, the “anion stabilizing group” refers to any suitable group that may stabilize the anion group via an intramolecular non-covalent bond, as provided herein. As such, the anion stabilizing group is configured to form an intramolecular non-covalent bond with the anion group, or, in other words, the anion stabilizing group is capable of forming an intramolecular non-covalent bond with the anion group. As used herein, the “non-covalent bond” may refer to any non-covalent bonding interaction between the anion stabilizing group and the anion group. As noted above, the non-covalent bonding interaction is intramolecular, where the anion stabilizing group and the anion group are on the same molecule. Exemplary non-covalent bonding includes hydrogen bonding or ionic bonding. The anion-stabilizing group may include a group that is protic. For example, the intramolecular non-covalent bond may be an intramolecular hydrogen bond between a suitable hydrogen atom of the anion stabilizing group and the anion group. For example, in some embodiments, the anion-stabilizing group may be configured to form an intramolecular hydrogen bond with the anion group, and, for example, in some embodiments, the anion-stabilizing group may form an intramolecular hydrogen bond with the anion group. In some embodiments, the intramolecular non-covalent bonding includes dipole-dipole interactions, ion-dipole interactions, or a combination thereof. As used herein, “non-covalent bond” does not include bonding based solely on Van der Waals forces.

In some embodiments, the anion-stabilizing group may have a pKa of 25 or less, typically 20 or less, or 18 or less, and preferably 16 or less.

In Formula (1), each Z¹ optionally further comprises one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, Z¹ may optionally further include one or more divalent linking groups selected from —O—, —C(O)—, —C(O)O—, —S(O)—, —S(O)₂—, —N(R′)—, —C(O)N(R′)—, substituted or unsubstituted C₁₋₁₀alkylene, substituted or unsubstituted C₃₋₁₀ cycloalkylene, substituted or unsubstituted C₃₋₁₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (1), two R¹ together optionally form a fused ring with ring A¹, wherein the fused ring optionally further comprises one or more divalent linking groups as part of its structure. Each of the one or more divalent linking groups is substituted or unsubstituted, and the fused ring is substituted or unsubstituted.

In Formula (1), one Z¹ and one R¹ together optionally may form a fused ring with ring A¹, wherein the fused ring optionally further comprises one or more divalent linking groups as part of its structure. Each of the one or more divalent linking groups is substituted or unsubstituted, and wherein the fused ring is substituted or unsubstituted. The fused ring that is formed with ring A¹ may be aliphatic or aromatic.

In Formula (1), each a and b is independently an integer from 0 to 2, provided that a sum of a and b is 1 or greater. Typically, a is 1 or 2, and b is 0.

In Formula (1), c is an integer from 0 to 10. Typically, c is an integer from 0 to 2, preferably c is 0 or 1.

In Formula (1), d is an integer from 1 to 3. Typically, d is 1 or 2, preferably d is 1. For example, in some embodiments, a may be 1 or 2 and d may be 1. In some embodiments, d may be 1 and L¹ is a single bond. It is to be understood that when L¹ is a single bond, then b is 0.

In Formula (1), e is 0 or 1. Typically, e is 0.

In Formula (1), n is an integer from 0 to 4. Typically, n is 0 or 1. Preferably, n is 0.

In some embodiments, the anion may be represented by Formula (1a):

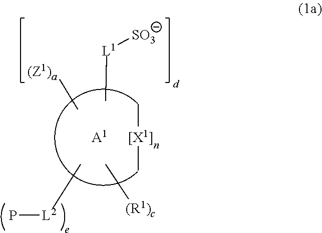

wherein ring A¹, X¹, R¹, Z¹, L¹, L², P, a, c, d, e, and n are each as defined for Formula (1).

In some embodiments, the anion may be represented by Formula (1b):

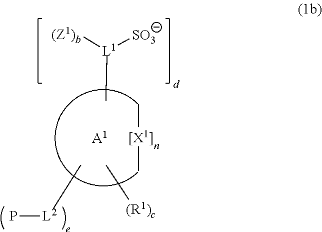

wherein ring A¹, X¹, R¹, Z¹, L¹, L², P, b, c, d, e, and n are each as defined for Formula (1).

Exemplary anions represented by Formula (1) include the following:

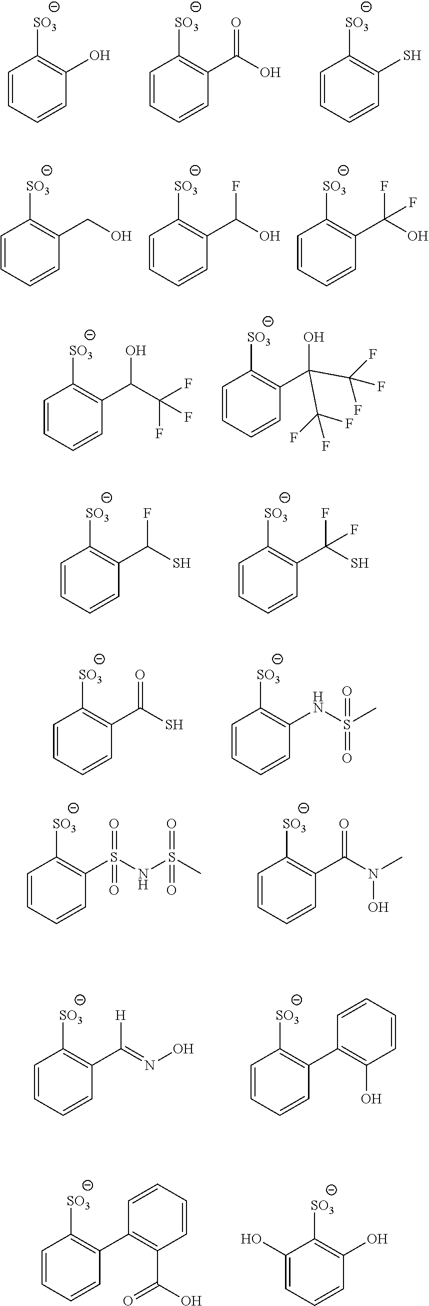

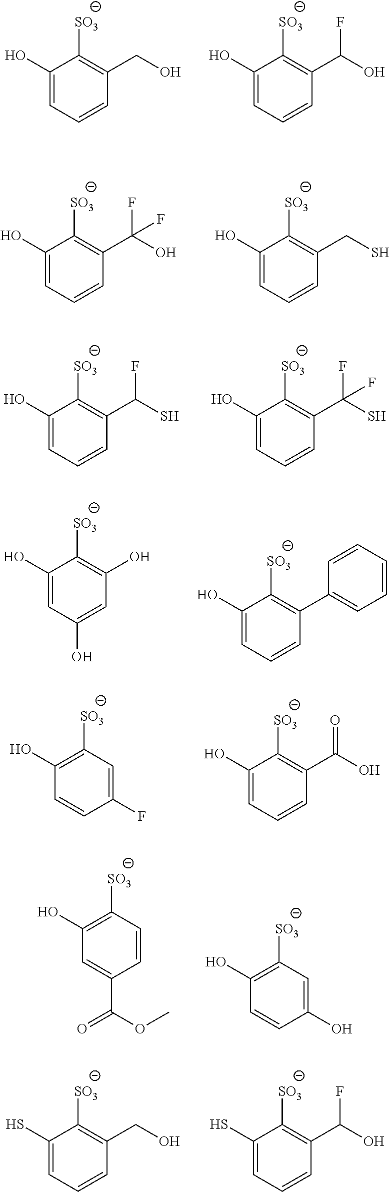

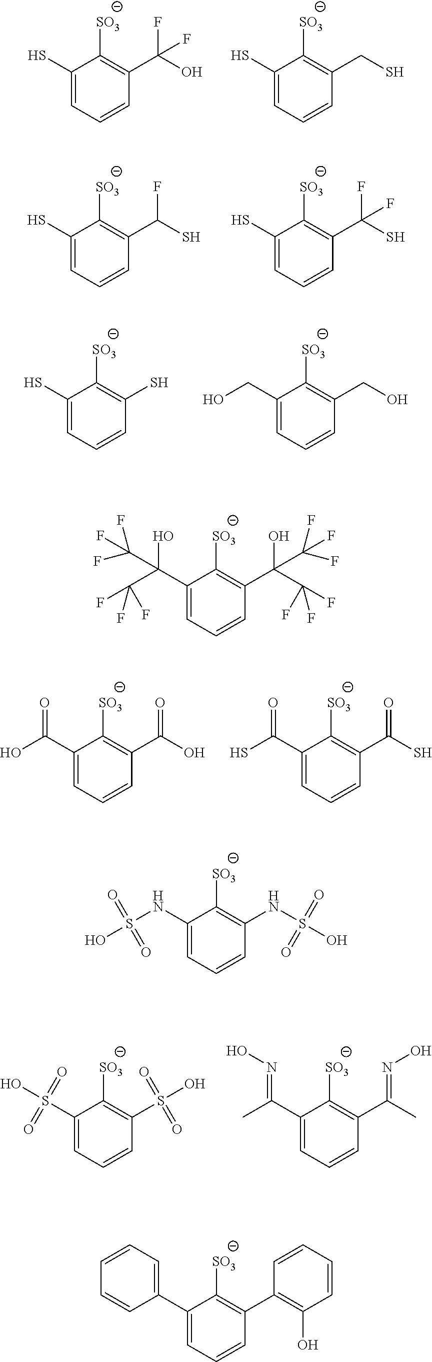

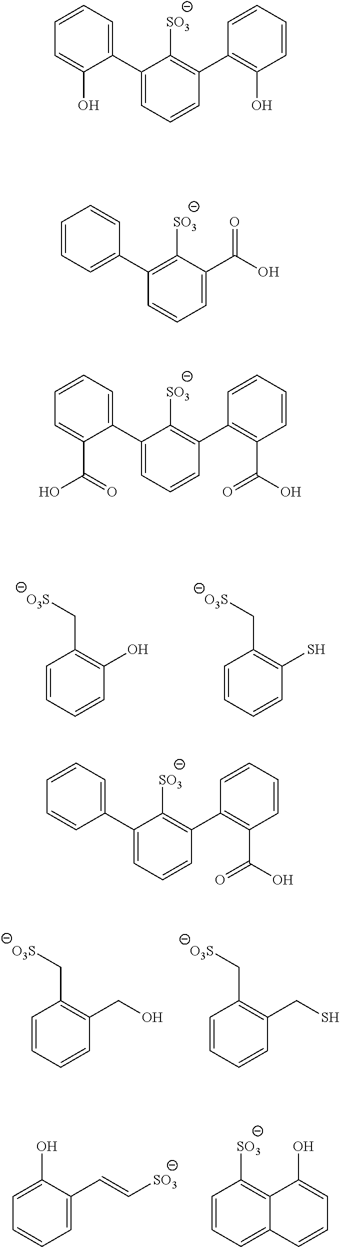

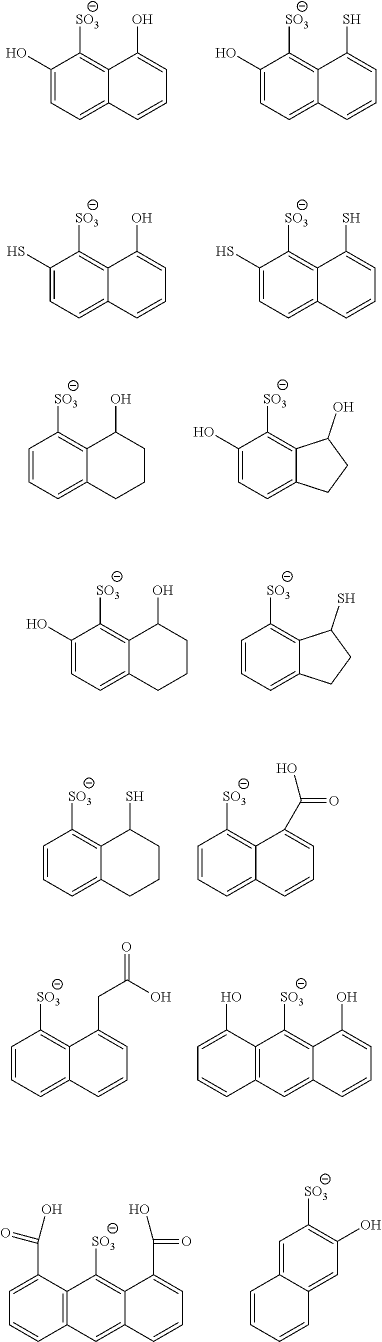

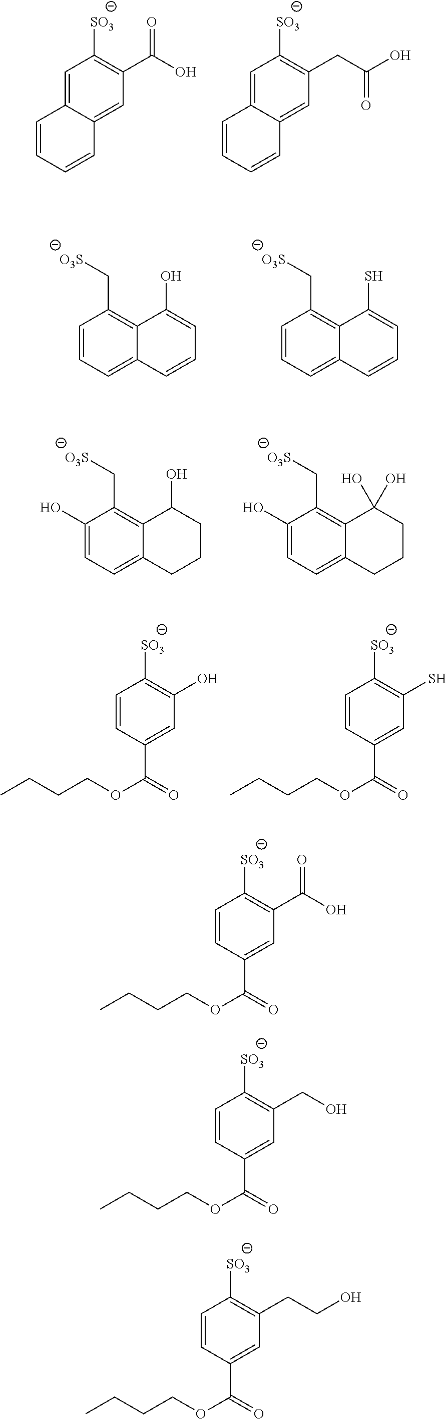

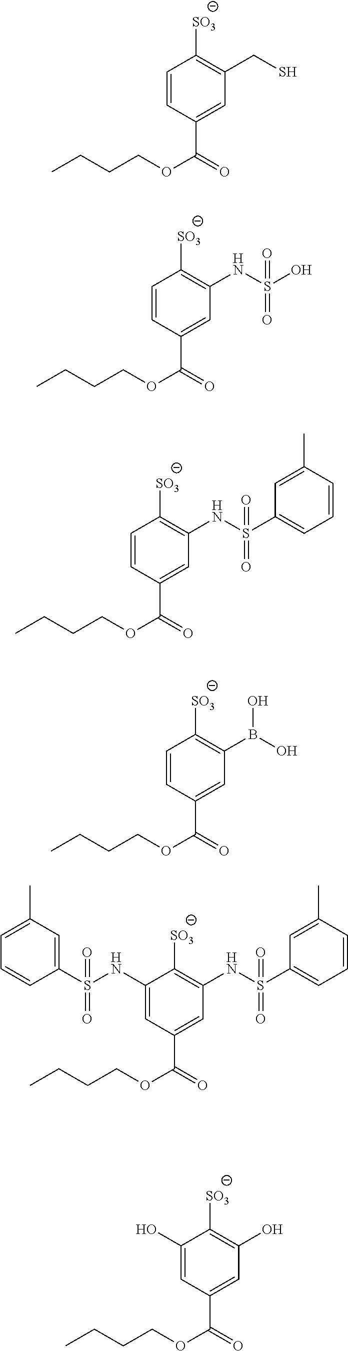

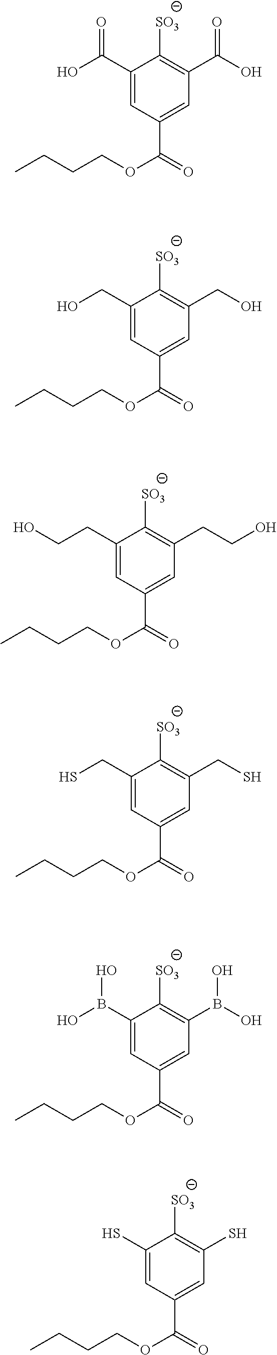

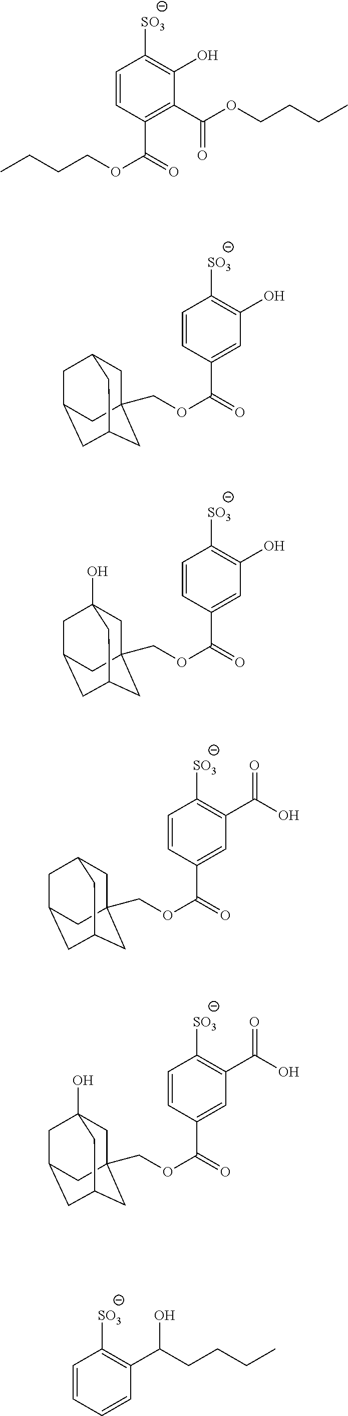

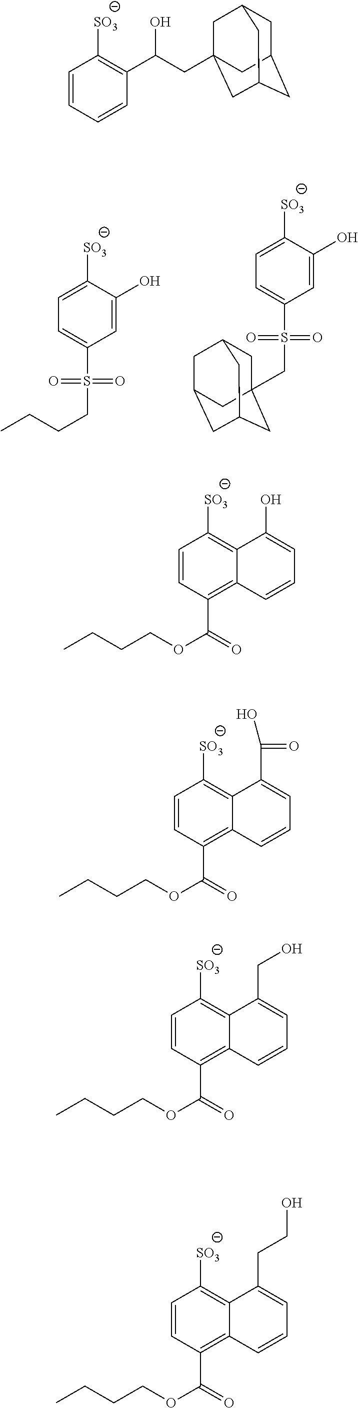

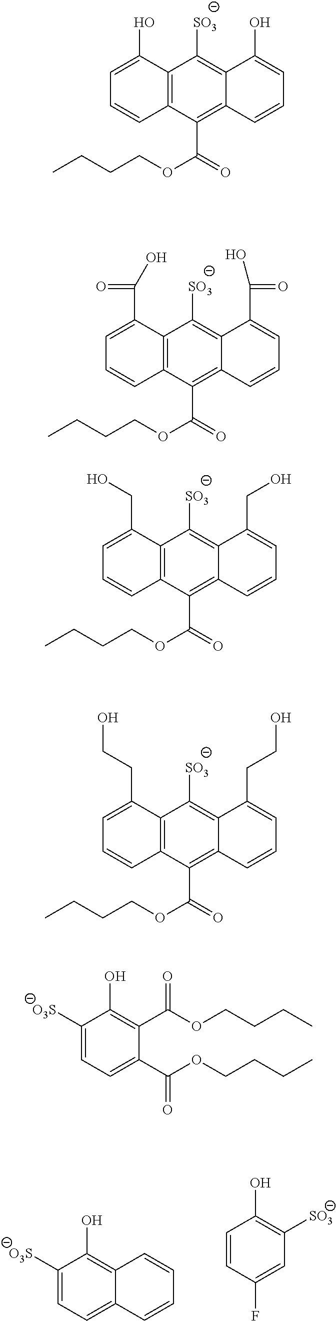

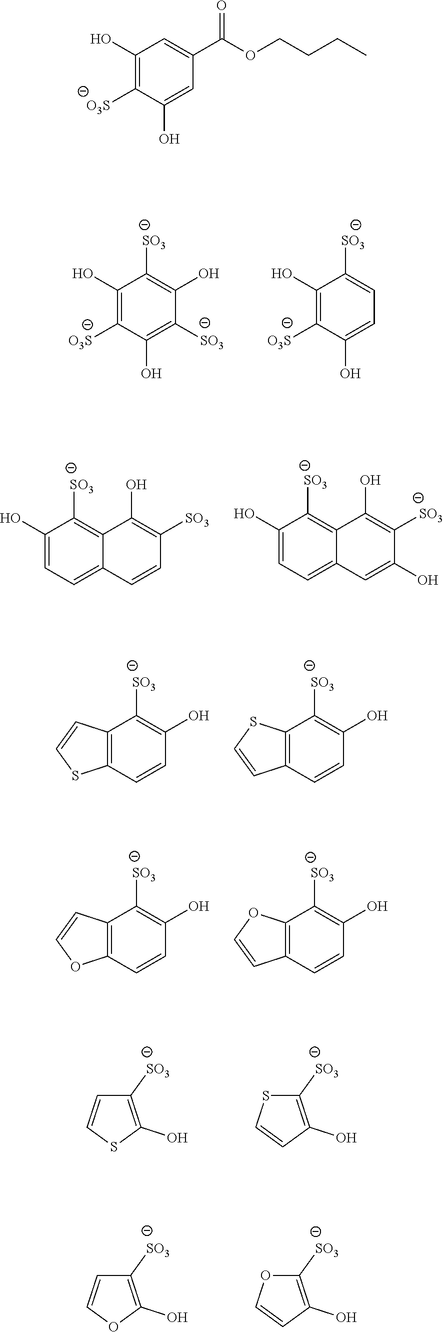

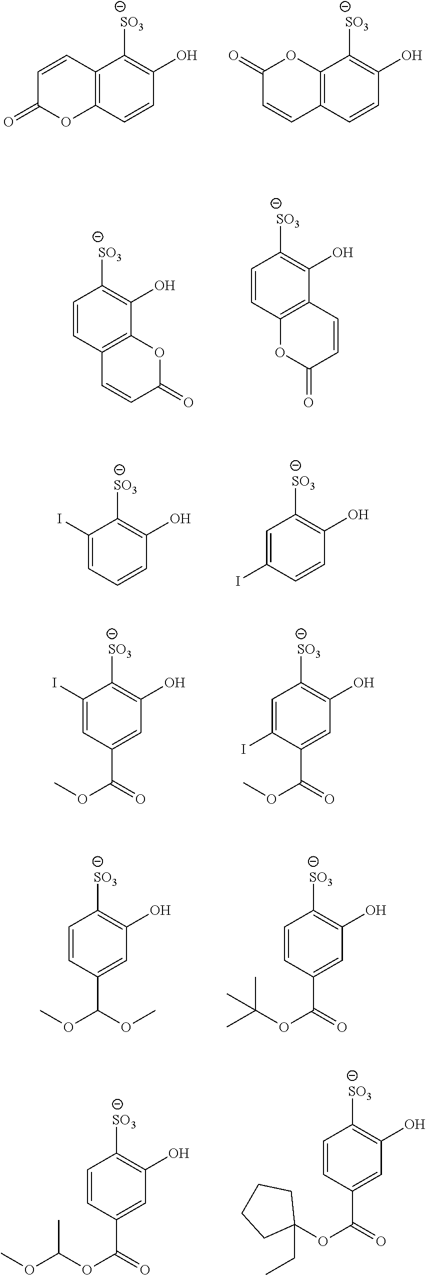

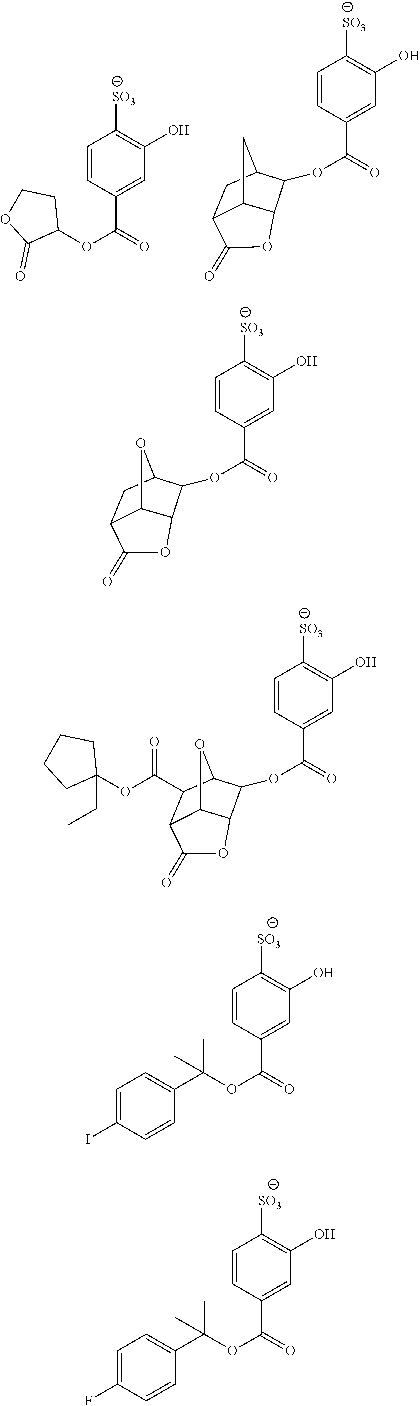

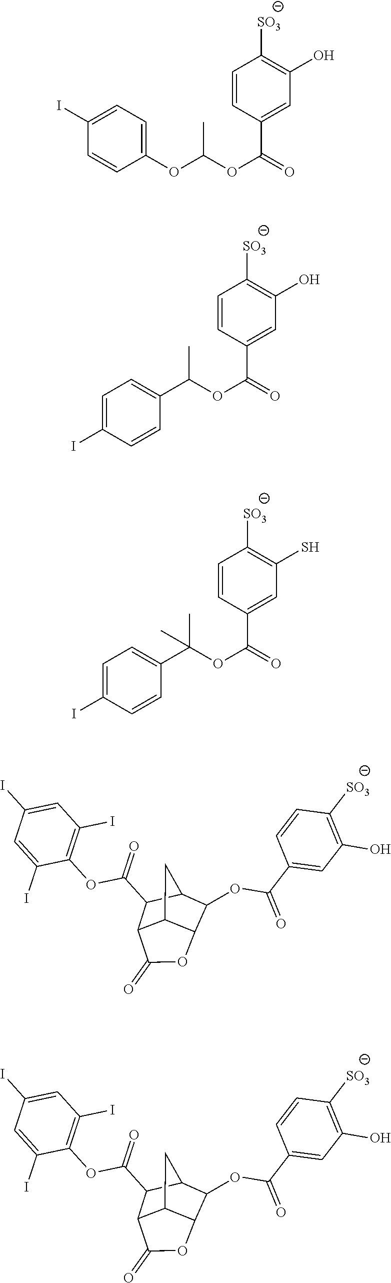

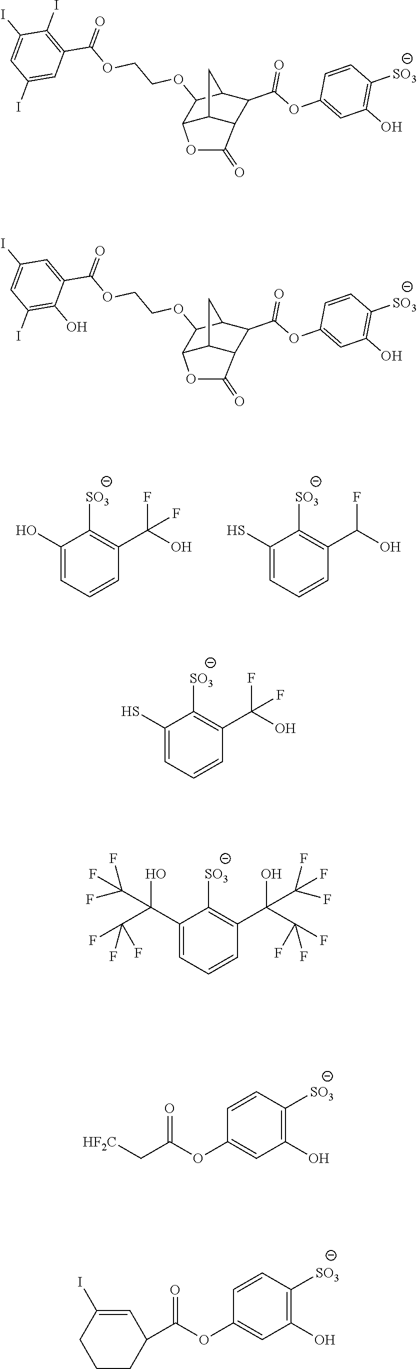

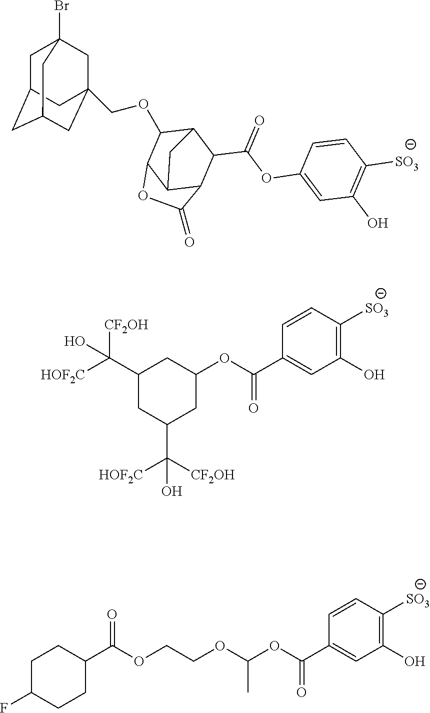

Other exemplary anions of Formula (1) include the following:

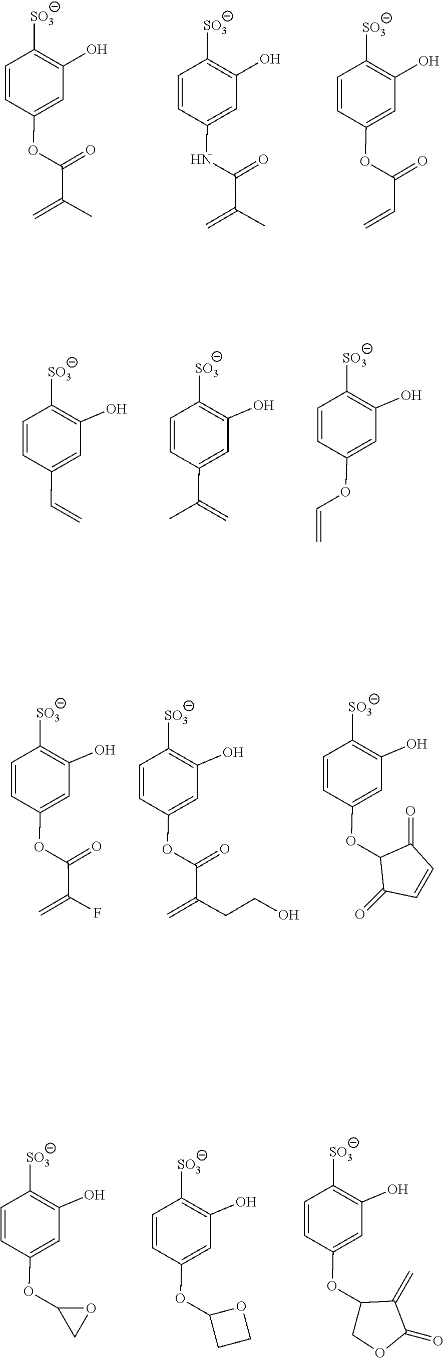

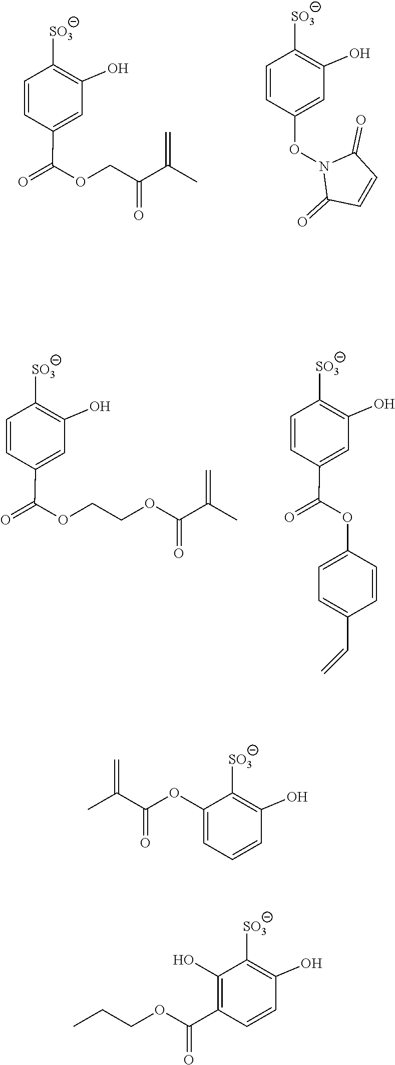

Other exemplary anions of Formula (1) include the following:

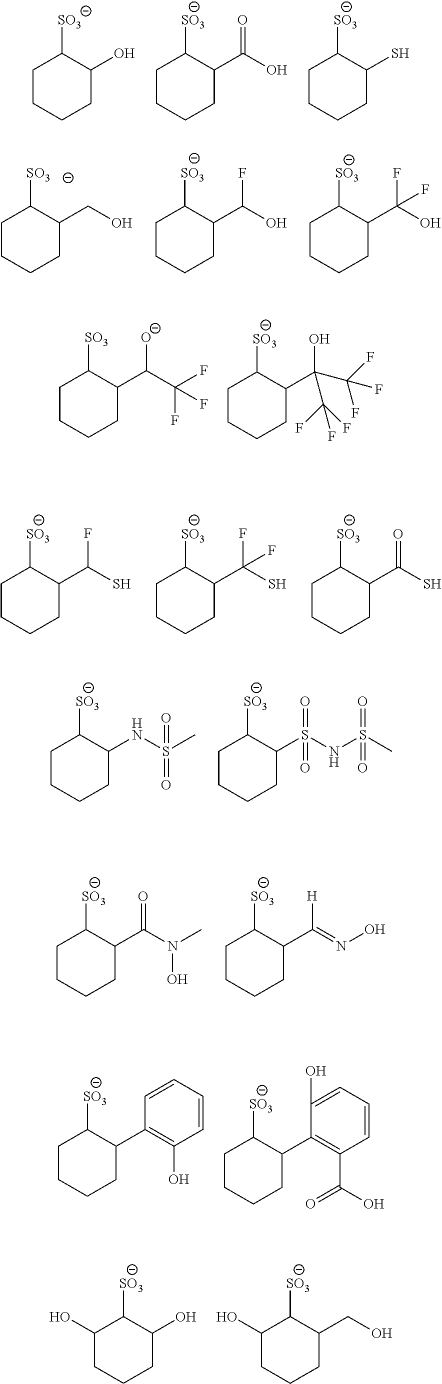

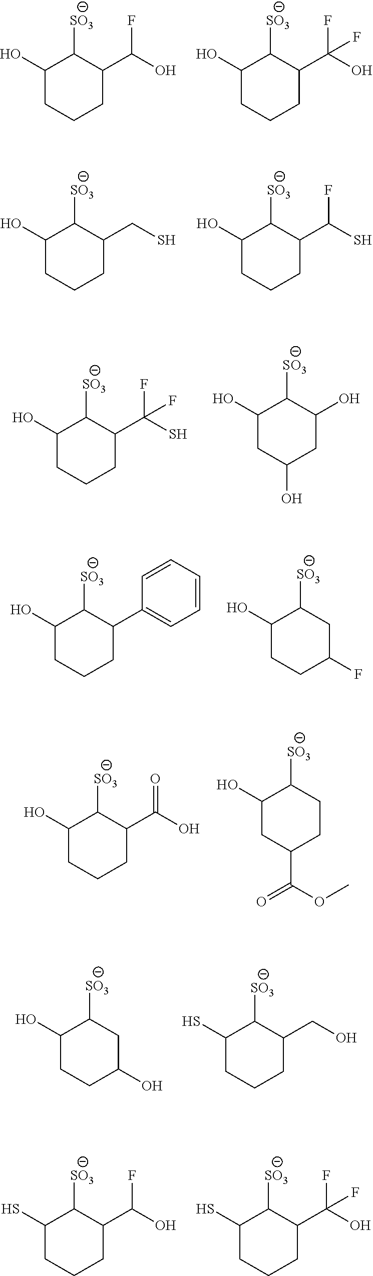

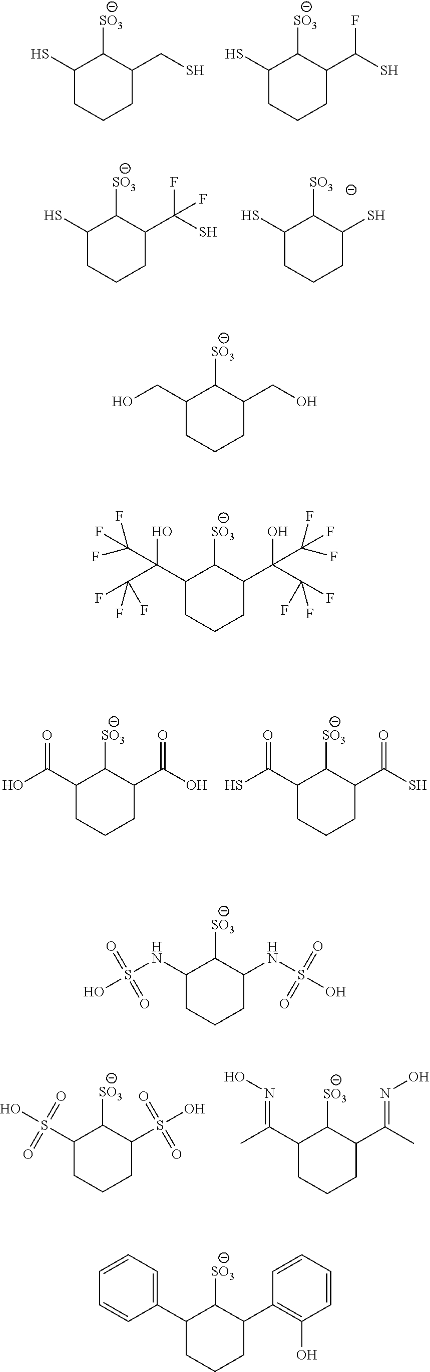

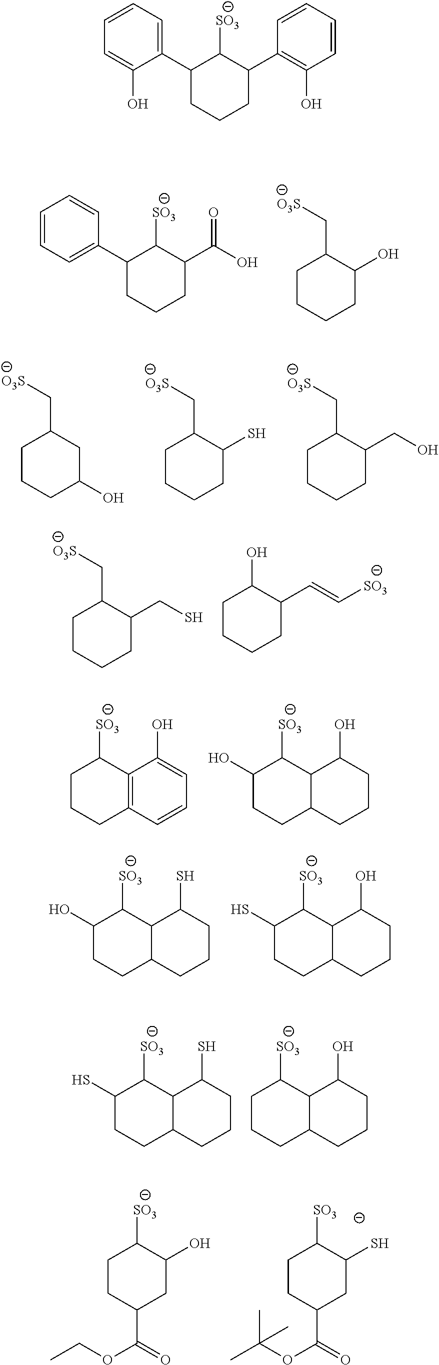

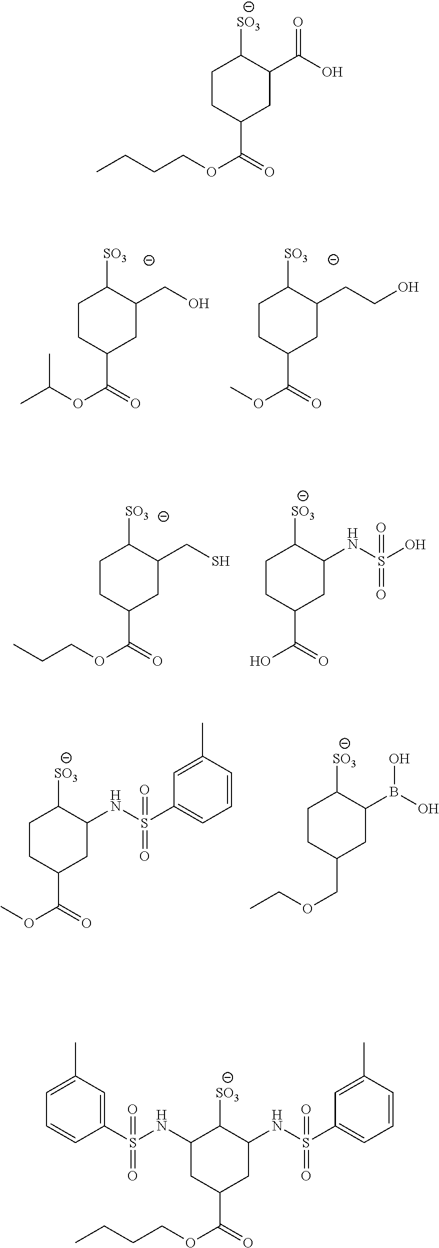

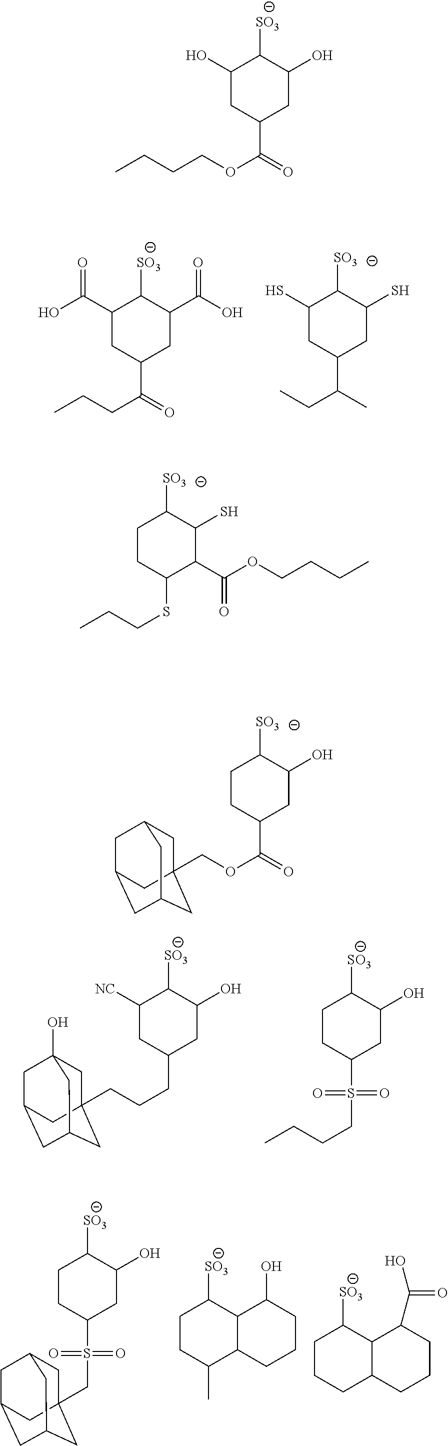

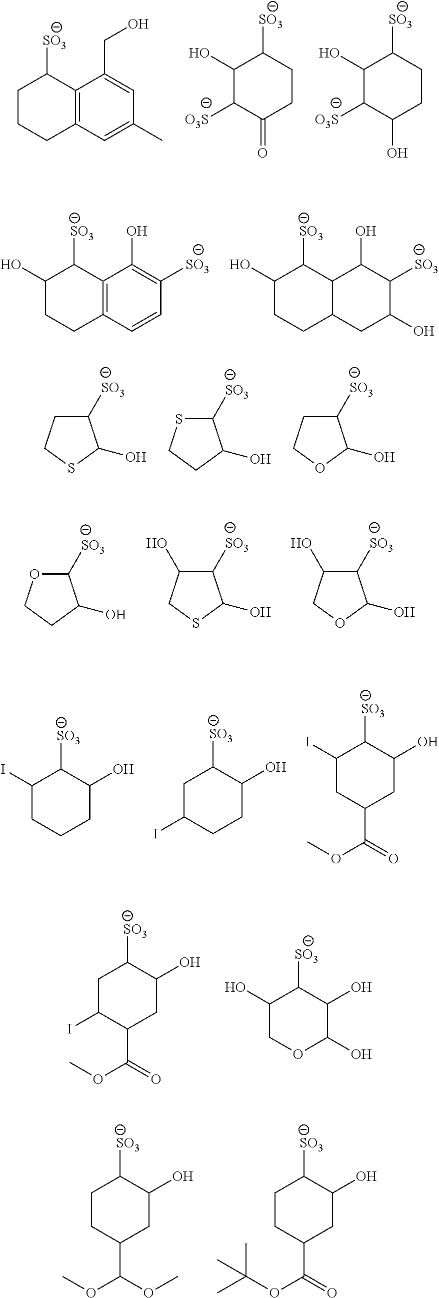

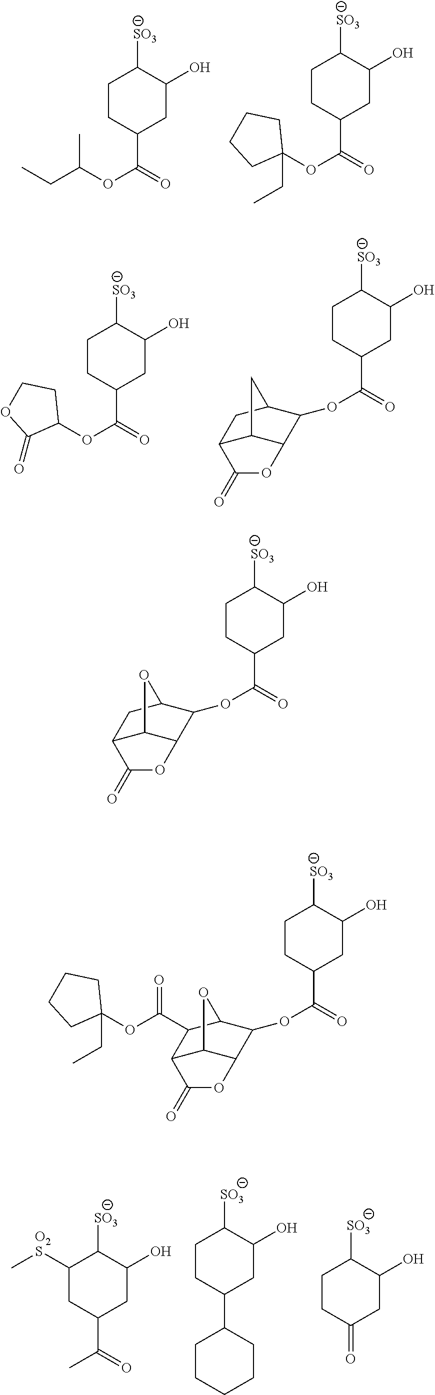

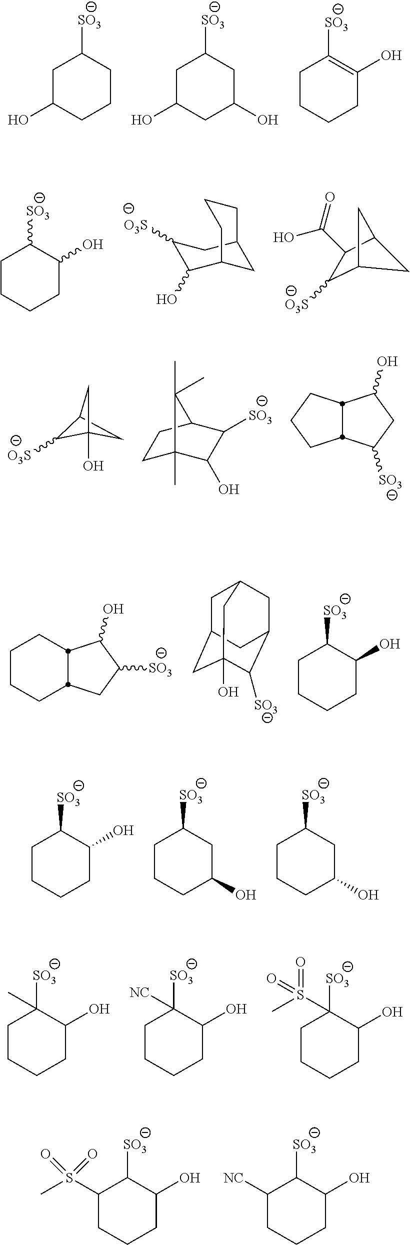

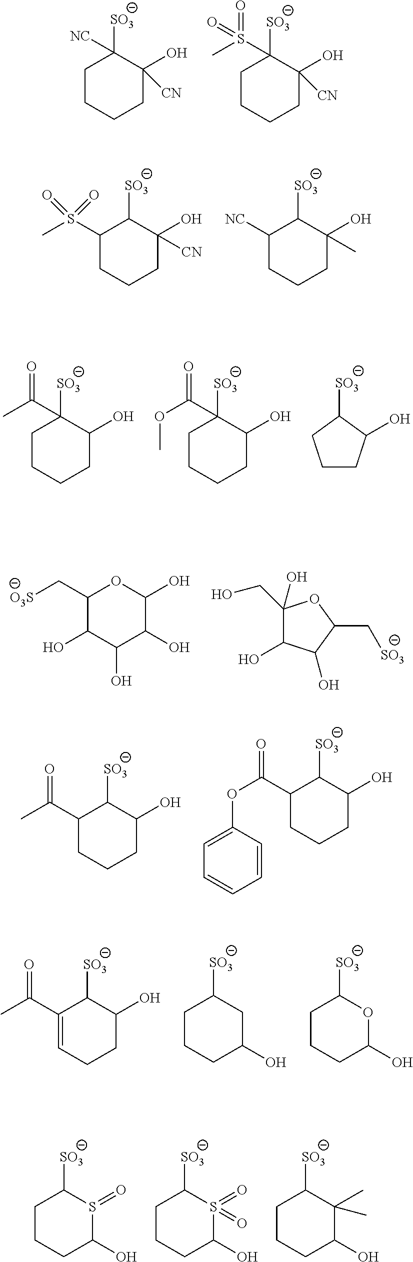

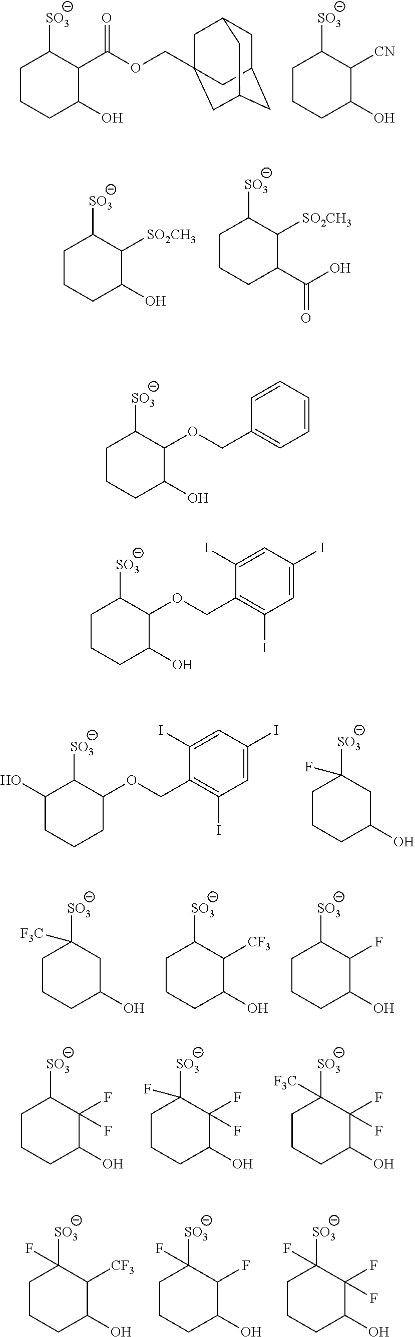

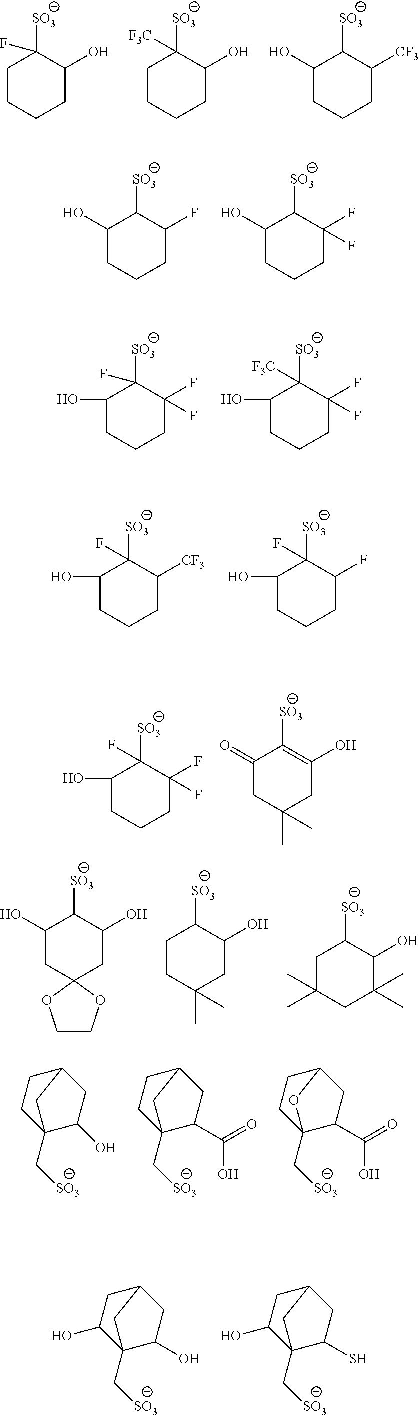

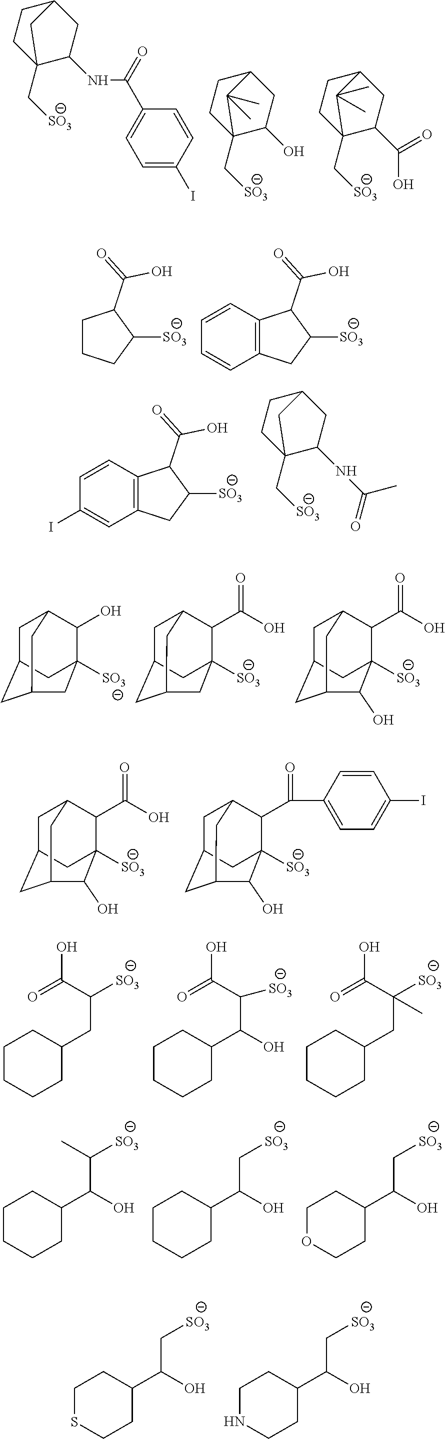

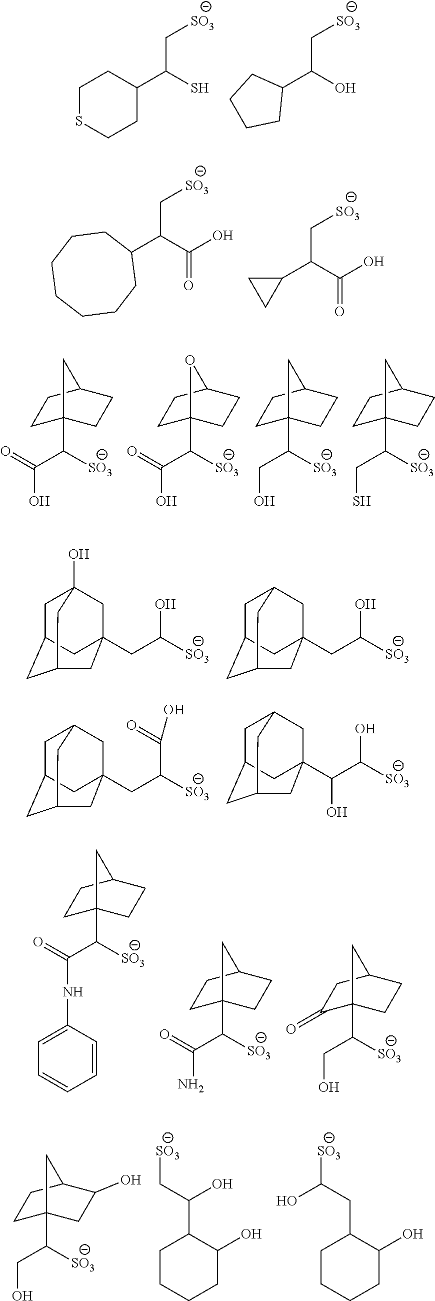

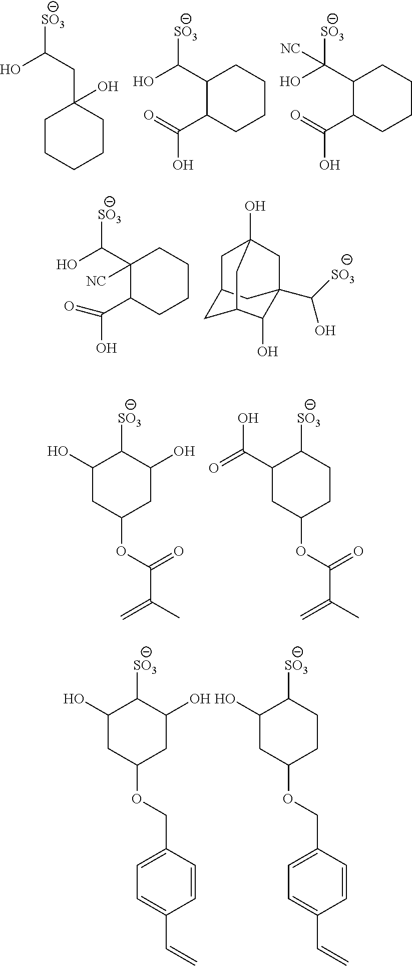

In some embodiments, a conjugate acid of the thermal acid generator compound may have a pKa of 0 or less. Typically, the conjugate acid of the thermal acid generator compound may have a pKa of −2 or less, preferably −5 or less. The conjugate acid of the thermal acid generator compound may, for example, have a pKa from −15 to 0 or from −15 to −2.

In some embodiments, the anion may be free of trifluoromethyl groups and difluoromethylene groups. In other words, in some embodiments, the anion of Formula (1) may be free of trifluoromethyl groups and difluoromethylene groups. For example, in some embodiments, the anion does not include fluorine (the anion of Formula (1) may be free of fluorine).

The anions of the thermal acid generator compound may be obtained from commercial sources or prepared by any suitable method. For example, such anions may be prepared as described in the Examples herein.

The thermal acid generator of Formula (1) further includes a nitrogen-containing cation. Exemplary cations include ammonium and pyridinium species, each of which may be optionally substituted with an acid-labile group, a lactone-containing group, a base-solubilizing group, a polymerizable group, or the like, or a combination thereof. Exemplary polymerizable groups include, but are not limited to, a (meth)acrylate group, an epoxy group, a vinyl aromatic group, a vinyl ether group, a vinyl ketone group, and/or a vinyl ester group.

In some embodiments, the nitrogen-containing cation may be of Formula (2) or (3):

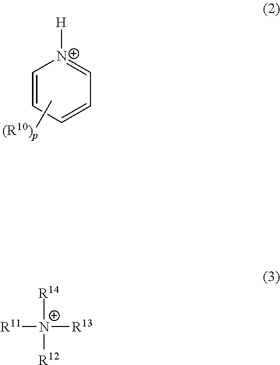

In Formula (2), each R¹⁰ is independently hydrogen or a non-hydrogen substituent, wherein R¹⁰ may further include one or more divalent linking groups as part of its structure. For example, each R¹⁰ may independently be hydrogen, deuterium, halogen, hydroxyl, substituted or unsubstituted C₁₋₃₀ alkyl, substituted or unsubstituted C₂₋₃₀ alkenyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy. Typically, each R¹⁰ may independently be hydroxyl, halogen, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy.

In Formula (2), each R¹⁰ optionally further includes one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In some embodiments, one or more R¹⁰ may each independently further include an acid-labile group, a lactone-containing group, a base-solubilizing group, a polymerizable group, or the like, or a combination thereof.

In Formula (2), p is an integer from 0 to 5. Typically, p is 1 or 2.

In Formula (3), R¹¹ to R¹⁴ are each independently hydrogen or a non-hydrogen substituent, wherein each of R¹¹ to R¹⁴ independently may further include one or more divalent linking groups as part of its structure. Typically, at least one of R¹¹ to R¹⁴ is not hydrogen or deuterium. For example, R¹¹ to R¹⁴ may each independently be hydrogen, deuterium, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ alkylheteroaryl, or substituted or unsubstituted C₄₋₃₀ heteroarylalkyl. Typically, R¹¹ to R¹⁴ may each independently be hydrogen, deuterium, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ alkylheteroaryl, or substituted or unsubstituted C₄₋₃₀ heteroarylalkyl.

In some embodiments, one or more of R¹¹ to R¹⁴ may each independently further include an acid-labile group, a lactone-containing group, a base-solubilizing group, a polymerizable group, or the like, or a combination thereof.

In Formula (3), each or R¹¹ to R¹⁴ may be either separate or connected to another group R¹¹ to R¹⁴ via a single bond or a divalent linking group to form a ring.

In some embodiments, the nitrogen-containing cation may be selected from the following:

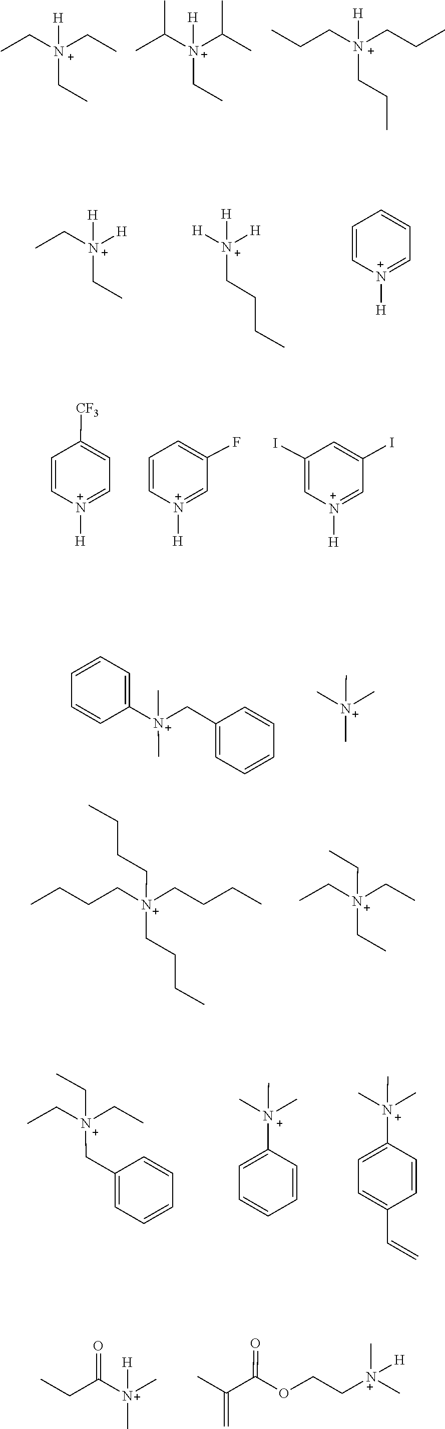

The cations for the thermal generator compounds may be obtained from commercial sources or prepared using common synthetic procedures.

Suitable thermal acid generator compounds include those resulting from any combination of the above-described anions and cations. The thermal acid generator compounds may be prepared by combining the anion and the cation species under appropriate conditions.

Also provided is a coating composition that includes the thermal acid generator, a polymer, and a solvent.

The thermal acid generator may be included in the coating composition in an amount from 0.1 to 99 weight percent (wt %), more typically from 1 to 80%, 1 to 75 wt %, or from 1 to 60 wt %, based on total solids of the coating composition. In some embodiments, the coating composition may include two or more different thermal acid generator compounds as described herein.

The coating composition includes a polymer, preferably a polymer that includes a crosslinkable group. Any suitable polymer that may be used to form an underlayer coating composition for a photoresist may be used. In some embodiments, the coating composition includes a polymer including a cyanurate structural unit, for example, derived from a compound of Formula (4):

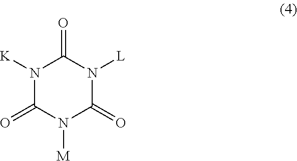

In Formula (4), K, L, and M are each independently a linear or branched C₁₋₁₀ hydrocarbon group, a C₁₋₁₀ alkoxycarbonyl group, a C₁₋₁₀ alkanoyloxy group, each of which is optionally substituted with a carboxylic acid group, or a linear or branched C₁₋₁₀ hydroxyalkyl group optionally substituted with a C₁₋₅ alkoxycarbonyl group or a C₁₋₅ substituted alkoxy group.

In Formula (4), for K, L, and M, each of the C₁₋₁₀ hydrocarbon group, the C₁₋₁₀ alkoxycarbonyl group, the C₁₋₁₀ alkanoyloxy group, and the C₁₋₁₀ hydroxyalkyl group may be optionally substituted with at least one of a halogen, an amino group, a thiol group, an epoxy group, an amido group, a C₁₋₅ alkyl group, a C₃₋₈ cycloalkyl group, a C₃₋₂₀ heterocycloalkyl group, a C₂₋₅ alkenyl group, a C₁₋₅ alkoxy group, a C₂₋₅ alkenoxy group, a C₆₋₁₂ aryl group, a C₆₋₁₂ aryloxy group, a C₇₋₁₃ alkylaryl group, or C₇₋₁₃ alkylaryloxy group. The C₃₋₈ cycloalkyl group and the C₃₋₂₀ heterocycloalkyl group may be optionally substituted on at least one ring carbon atom with an oxo group (═O). At least one hydrogen atom of the polymer derived from the monomer of Formula (4) may be substituted with a functional group independently chosen from hydroxyl, carboxyl, thiol, amino, epoxy, alkoxy, amido, vinyl, and a combination thereof. Of these, hydroxyl, carboxyl, or alkoxy are preferred.

The polymer may be formed by conventional polycondensation techniques such as for example described in Zeno W. Wicks, Jr, Frank N. Jones, S. Peter Pappas, “Organic Coatings, Science and Technology,” pp 246-257 (John Wiley & Sons, 1999, second edition) and references therein or in Houben-Weyl, “Methoden der Organischen Chemie, Band E20, Makromolekulare Soffe, Polyester,” pp 1405-1429. (Georg Thieme Verlag, Stuttgart 1987) and references therein. In one aspect, a diol or polyol and a dicarboxylic acid or polycarboxylic acid are charged into a conventional polymerization vessel and reacted between about 150 and 280° C. for several hours. Optionally, an esterification catalyst may be used to decrease the reaction time. It is also understood that an esterifiable derivative of a polycarboxylic acid, such as a dimethyl ester or anhydride of a polycarboxylic acid, can be used to prepare the polyester. Exemplary polyols and polycarboxylic acids include isocyanurate polyols and isocyanurate polycarboxylic acids. The polyester polymers can be linear or branched. Examples of the polymer including cyanurate structural units are also provided in U.S. Patent Publication No. 2011/0033801 A1 and U.S. Patent Publication No. 2019/0129305 A1, the contents of which are incorporated herein in their entireties by reference.

Suitable dicarboxylic acids or polycarboxylic acids, or their corresponding alkyl esters, that may be used to form the second polymer include saturated as well as unsaturated dicarboxylic acids such as, for example, isophthalic acid, maleic acid, maleic anhydride, malonic acid, fumaric acid, succinic acid, succinic anhydride, glutaric acid, adipic acid, 2-methyl-1,6-hexanoic acid, pimelic acid, suberic acid, dodecanedioic acids, phthalic acid, phthalic anhydride, 5-tert butyl isophthalic acid, tetrahydrophthalic anhydride, hexahydrophthalic acid, hexahydrophthalic anhydride, endomethylenetetrahydrophthalic anhydride, azelaic acid, sebacic acid, tetrachlorophthalic anhydride, chlorendic acid, isophthalic acid, trimellitic anhydride, terephthalic acid, naphthalene dicarboxylic acid, cyclohexane-dicarboxylic acid, dimer fatty acid, or anhydrides of any of these acids, or combinations thereof.

Suitable diols and polyols include, but are not limited to, ethylene glycol, diethylene glycol, triethylene glycol and higher polyethylene glycols, propylene glycol, dipropylene glycol, tripropylene glycol and higher polypropylene glycols, 1,3-propanediol, 1,4-butanediol and other butanediols, 1,5-pentanediol and other pentane diols, hexanediols, decanediols, and dodecanediols, glycerol, trimethylolpropane, trimethylolethane, neopentyl glycol, pentaerythritol, cyclohexanedimethanol, dipentaerythtritol, 1,2-methyl-1,3-propanediol, 1,4-benzyldimethanol, 2,4-dimethyl-2-ethylhexane-1,3-diol, isopropylidene bis (p-phenylene-oxypropanol-2), 4,4′-dihydroxy-2,2′-diphenylpropane, 1,3-cyclohexanedimethanol, 1,4-cyclohexanedimethanol (or mixtures of 1,3 and 1,4 cyclohexanedimethanol, may be cis or trans), sorbitol, or the like, or combinations thereof.

The polymer may optionally include one or more additional repeating unit(s) different from the repeating unit comprising Formula (4). The additional repeating units may include, for example, one or more additional units for purposes of adjusting properties of the coating composition, such as etch rate and solubility. Exemplary additional units may include one or more of (meth)acrylate, vinyl ether, vinyl ketone, and vinyl ester. The one or more additional repeating units if present in the polymer are typically used in an amount of up to 99 mol %, and typically from 3 to 80 mol %, based on total repeating units of the polymer.

Preferably, the polymer will have a weight average molecular weight (Mw) of 1,000 to 100,000 grams per mole (g/mol), more typically 2,000 to 30,000 g/mol, and a number average molecular weight (Mn) of 500 to 1,000,000 g/mol. Molecular weights (either MW or Mn) are suitably determined by gel permeation chromatography (GPC).

The amount of the polymer in the coating composition may be from 15 wt % to 99 wt % based on total solids of the coating composition. For example, the amount of the polymer in the coating composition may be from 15 wt % to 95 wt % based on total solids of the coating composition, but is not limited thereto.

The coating composition may further include a crosslinking agent, which is not specifically limited and may be any crosslinking substance that may initiate a crosslinking reaction by heat. The crosslinkable compound and the polymer may be cured, crosslinked, or hardened when the thermal acid generator releases an acid upon exposure to heat.

Any suitable crosslinking agent may be used in the present compositions, provided that such crosslinking agent has at least two, and preferably at least three, moieties capable of reacting with the present polymer under suitable conditions, such as under acidic conditions. Exemplary crosslinking agents include, but are not limited to, novolac resins, epoxy-containing compounds, melamine compounds, guanamine compounds, isocyanate-containing compounds, benzocyclobutenes, benzoxazines, and the like, and typically any of the foregoing having two or more, more typically three or more substituents selected from methylol, C₁₋₁₀ alkoxymethyl, and C₂₋₁₀ acyloxymethyl. Examples of suitable crosslinking agents include the following:

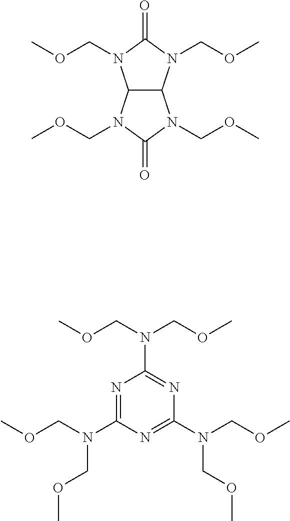

The amount of the crosslinking agent in the coating composition may be from 0 wt % to 15 wt % by weight based on total solids of the coating composition. For example, the amount of the crosslinking agent in the coating composition may be from 0 wt % to 10 wt % based on total solids of the coating composition or from 0 wt % to 5 wt % based on total solids of the coating composition, but is not limited thereto.

The coating composition further includes a solvent. Preferably, the solvent is an organic solvent conventionally used in the manufacture of electronic devices. Suitable solvents include, for example: aliphatic hydrocarbons such as hexane and heptane; aromatic hydrocarbons such as toluene and xylene; halogenated hydrocarbons such as dichloromethane, 1,2-dichloroethane and 1-chlorohexane; alcohols such as methanol, ethanol, 1-propanol, iso-propanol, tert-butanol, 2-methyl-2-butanol, 4-methyl-2-pentanol, and diacetone alcohol (4-hydroxy-4-methyl-2-pentanone) (DAA); propylene glycol monomethyl ether (PGME); ethers such as diethyl ether, tetrahydrofuran, 1,4-dioxane, and anisole; ketones such as acetone, methyl ethyl ketone, methyl iso-butyl ketone, 2-heptanone, and cyclohexanone (CHO); esters such as ethyl acetate, n-butyl acetate, propylene glycol monomethyl ether acetate (PGMEA), ethyl lactate (EL), hydroxyisobutyrate methyl ester (HBM), and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, propylene carbonate, diphenyl carbonate, and propylene carbonate; polar aprotic solvents such as dimethyl sulfoxide and dimethyl formamide; water; and combinations thereof. Of these, preferred solvents include one or more of PGME, PGMEA, EL, GBL, HBM, CHO, DAA, or a combination thereof.

The total solvent content (i.e., cumulative solvent content for all solvents) in the compositions is typically from 40 to 99 wt %, for example, from 60 to 99 wt %, or from 85 to 99 wt %, based on total weight of the coating composition. The desired solvent content will depend, for example, on the desired thickness of the coated layer and the coating conditions.

Additionally, the coating compositions may further include one or more polymers in addition to and different from the polymer as described above. For example, the coating compositions may include an additional polymer as described above but different in composition. Additionally, or alternatively, the one or more additional polymers may include those well known in the photoresist art, for example, those chosen from polyacrylates, polyvinylethers, polyesters, polynorbornenes, polyacetals, polyethylene glycols, polyamides, polyacrylamides, polyphenols, novolacs, styrenic polymers, polyvinyl alcohols, or combinations thereof.

The coating composition may further include one or more additional, optional additives. For example, optional additives may include actinic and contrast dyes, anti-striation agents, plasticizers, speed enhancers, sensitizers, photo-decomposable quenchers (PDQ) (and, also known as photo-decomposable bases), basic quenchers, surfactants, and the like, or combinations thereof. If present, the optional additives are typically present in the compositions in an amount of from 0.01 to 10 wt %, based on total solids of the composition.

The coating composition may optionally include one or more surface leveling agents (or surfactants). Typical surfactants include those which exhibit an amphiphilic nature, meaning that they may be both hydrophilic and hydrophobic at the same time. Amphiphilic surfactants possess a hydrophilic head group or groups, which have a strong affinity for water and a long hydrophobic tail, which is organophilic and repels water. Suitable surfactants may be ionic (i.e., anionic, cationic) or nonionic. Further examples of surfactants include silicone surfactants, poly(alkylene oxide) surfactants, and fluorochemical surfactants. Suitable non-ionic surfactants include, but are not limited to, octyl and nonyl phenol ethoxylates such as TRITON X-114, X-100, X-45, X-15 and branched secondary alcohol ethoxylates such as TERGITOL TMN-6 (The Dow Chemical Company, Midland, Michigan USA) and PF-656 (Omnova Solutions, Beachwood, Ohio, USA). Still further exemplary surfactants include alcohol (primary and secondary) ethoxylates, amine ethoxylates, glucosides, glucamine, polyethylene glycols, poly(ethylene glycol-co-propylene glycol), or other surfactants disclosed in McCutcheon's Emulsifiers and Detergents, North American Edition for the Year 2000 published by Manufacturers Confectioners Publishing Co. of Glen Rock, N.J. Nonionic surfactants that are acetylenic diol derivatives also may be suitable. Such surfactants are commercially available from Air Products and Chemicals, Inc. of Allentown, PA and sold under the trade names of SURFYNOL and DYNOL. Additional suitable surfactants include other polymeric compounds such as the tri-block EO-PO-EO co-polymers PLURONIC 25R2, L121, L123, L31, L81, L101, and P123 (BASF, Inc.). Such surfactants if used may be present in the coating composition in minor amounts, for example from greater than 0 to 1 wt % based on total solids of the coating composition.

Another aspect provides a coated substrate, including a cured layered of the coating composition disposed on a substrate; and a photoresist layer disposed on the layer of the coating composition. As used herein, the term “cured layer” refers to a layer derived from the coating composition after the composition has been disposed on a substrate and subsequently cured to form a coating layer or film. In other words, curing the coating composition forms a cured layer derived from the coating composition.

Still another aspect of the present invention provides a method of forming a pattern. The method includes: applying a layer of the inventive coating composition on a substrate; curing the layer of the coating composition to form an underlayer film; applying a layer of a photoresist composition on the underlayer film to form a photoresist layer; pattern-wise exposing the photoresist layer to activating radiation; and developing the exposed photoresist layer to provide a resist relief image.

A wide variety of substrates may be used in the patterning methods, with electronic device substrates being typical. Suitable substrates include, for example, packaging substrates such as multichip modules; flat panel display substrates; integrated circuit substrates; substrates for light emitting diodes (LEDs) including organic light emitting diodes (OLEDs); semiconductor wafers; polycrystalline silicon substrates; and the like. Suitable substrates may be in the form of wafers such as those used in the manufacture of integrated circuits, optical sensors, flat panel displays, integrated optical circuits, and LEDs. As used herein, the term “semiconductor wafer” is intended to encompass “an electronic device substrate,” “a semiconductor substrate,” “a semiconductor device,” and various packages for various levels of interconnection, including a single-chip wafer, multiple-chip wafer, packages for various levels, or other assemblies requiring solder connections. Such substrates may be any suitable size. Typical wafer substrate diameters are 200 mm to 300 mm, although wafers having smaller and larger diameters may be suitably employed according to the present invention. As used herein, the term “semiconductor substrate” includes any substrate having one or more semiconductor layers or structures which may optionally include active or operable portions of semiconductor devices. A semiconductor device refers to a semiconductor substrate upon which at least one microelectronic device has been or is being batch fabricated.

The substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. The substrate may include one or more layers and patterned features. The layers may include, for example, one or more conductive layers such as layers of aluminum, copper, molybdenum, tantalum, titanium, tungsten, alloys, nitrides or silicides of such metals, doped amorphous silicon or doped polysilicon, one or more dielectric layers such as layers of silicon oxide, silicon nitride, silicon oxynitride, or metal oxides, semiconductor layers, such as single-crystal silicon, and combinations thereof. The layers can be formed by various techniques, for example, chemical vapor deposition (CVD) such as plasma-enhanced CVD (PECVD), low-pressure CVD (LPCVD) or epitaxial growth, physical vapor deposition (PVD) such as sputtering or evaporation, or electroplating.

It may be desired in certain patterning methods of the invention to provide one or more lithographic layers such as a hardmask layer, for example, a spin-on-carbon (SOC), amorphous carbon, or metal hardmask layer, a CVD layer such as a silicon nitride (SiN) layer, silicon oxide (SiO) layer, or silicon oxynitride (SiON) layer, an organic or inorganic BARC layer, or a combination thereof, on an upper surface of the substrate prior to forming the photoresist underlayer (coating layer) of the invention. Such layers, together with an overcoated underlayer of the invention and photoresist layer, form a lithographic material stack. Typical lithographic stacks which may be used in the patterning methods of the invention include, for example, the following: SOC layer/underlayer/photoresist layer; SOC layer/SiON layer/underlayer/photoresist layer; SOC layer/SiARC layer/underlayer/photoresist layer; SOC layer/metal hardmask layer/underlayer/photoresist layer; amorphous carbon layer/underlayer/photoresist layer; and amorphous carbon layer/SiON layer/underlayer/photoresist layer.

The coating composition may be coated on the substrate by any suitable means, such as spin-coating, slot-die coating, doctor blading, curtain-coating, roller-coating, spray-coating, dip-coating, and the like. In the case of a semiconductor wafer, spin-coating is preferred. In a typical spin-coating method, the coating compositions are applied to a substrate which is spinning at a rate of 500 to 4000 rpm for a period of 15 to 90 seconds to obtain a desired layer of the coating composition on the substrate. It will be appreciated by those skilled in the art that the thickness of the coated composition may be adjusted by changing the spin speed, as well as the solids content of the coating composition. An underlayer formed from the coating composition typically has a dried layer thickness of from 1 to 50 nm, more typically from 1 to 20 nm.

The applied coating composition is optionally softbaked at a relatively low temperature to remove any solvent and other relatively volatile components from the coating composition. Typically, the coated substrate is baked at a temperature of less than or equal to 150° C., preferably from 60 to 130° C., and more preferably from 90 to 120° C. The baking time is typically from 10 seconds to 10 minutes, preferably from 30 seconds to 5 minutes, and more preferably from 6 to 120 seconds. When the substrate is a wafer, such baking steps may be performed by heating the wafer on a hot plate. The soft-baking step may be performed as part of the curing of the applied coating composition or may be omitted altogether.

The applied coating composition is then cured to form a photoresist underlayer film. The applied photoresist underlayer composition should be sufficiently cured such that the resulting underlayer does not intermix, or minimally intermixes, with a subsequently applied layer, such as a photoresist or other organic or inorganic layer disposed directly on the photoresist underlayer. The applied photoresist underlayer composition may be cured in an oxygen-containing atmosphere, such as air, or in an inert atmosphere, such as nitrogen and under conditions, such as heating, sufficient to provide a cured coating layer. This curing step is preferably conducted on a hot plate-style apparatus, although oven curing may be used to obtain equivalent results. The curing temperature should be sufficient for the TAG to effect curing throughout the layer, for example, sufficient to allow the thermal acid generator to liberate acid and the liberated acid to effect crosslinking. Typically, the curing is conducted at a temperature of 150° C. or greater, and preferably 150 to 450° C. It is more preferred that the curing temperature is 180° C. or greater, still more preferably 200° C. or greater, and even more preferably from 200 to 400° C. The curing time is typically from 10 seconds to 10 minutes, preferably from 30 seconds to 5 minutes, more preferably from 45 seconds to 2 minutes, and still more preferably from 45 to 90 seconds. Optionally, a ramped or a multi-stage curing process may be used. A ramped bake typically begins at a relatively low (e.g., ambient) temperature that is increased at a constant or varied ramp rate to a higher target temperature. A multi-stage curing process involves curing at two or more temperature plateaus, typically a first stage at a lower bake temperature and one or more additional stages at a higher temperature. Conditions for such ramped or multi-stage curing processes are known to those skilled in the art and may allow for omission of a preceding softbake process.

After curing of the applied photoresist underlayer composition to form an underlayer film, one or more processing layers, such as a photoresist layer, a hardmask layer such as a metal hardmask layer, an organic or inorganic BARC layer, and the like, may be disposed over the cured photoresist underlayer film. A photoresist layer may be formed directly on the surface of the photoresist underlayer or, alternatively, may be formed above the photoresist underlayer on one or more intervening layers. In this case, one or more intervening processing layers such as described above can be sequentially formed over the photoresist underlayer followed by formation of the photoresist layer. Determination of suitable layers, thicknesses and coating methods are well known to those skilled in the art.

A wide variety of photoresists may be suitably used in the methods of the invention and are typically positive-tone materials. Suitable photoresists include, for example, materials within the EPIC series of photoresists available from DuPont Electronics & Imaging (Marlborough, Massachusetts). The photoresist can be applied to the substrate by known coating techniques such as described above with reference to the photoresist underlayer composition, with spin-coating being typical. A typical thickness for the photoresist layer is from 10 to 300 nm. The photoresist layer is typically next softbaked to minimize the solvent content in the layer, thereby forming a tack-free coating and improving adhesion of the layer to the substrate. The softbake can be conducted on a hotplate or in an oven, with a hotplate being typical. Typical softbakes are conducted at a temperature of from 70 to 150° C., and a time of from 30 to 90 seconds.

The photoresist layer is next exposed to activating radiation through a photomask to create a difference in solubility between exposed and unexposed regions. References herein to exposing a photoresist composition to radiation that is activating for the composition indicates that the radiation can form a latent image in the photoresist composition. The photomask has optically transparent and optically opaque regions corresponding to regions of the resist layer to be exposed and unexposed, respectively, by the activating radiation. The exposure wavelength is typically sub-400 nm, and more typically, sub-300 nm, such as 248 nm (KrF), 193 nm (ArF), or an EUV wavelength (e.g., 13.5 nm), or an e-beam. In a preferred aspect, the exposure wavelength is 193 nm or an EUV wavelength. The exposure energy is typically from 10 to 150 mJ/cm², depending, for example, on the exposure tool and the components of the photosensitive composition.

Following exposure of the photoresist layer, a post-exposure bake (PEB) is typically performed. The PEB can be conducted, for example, on a hotplate or in an oven. The PEB is typically conducted at a temperature of from 70 to 150° C., and a time of from 30 to 90 seconds. A latent image defined by the boundary between polarity-switched and unswitched regions (corresponding to exposed and unexposed regions, respectively) is thereby formed. The exposed photoresist layer is then developed using the appropriate developer to provide a patterned photoresist layer.

The pattern of the photoresist layer can then be transferred to one or more underlying layers including the photoresist underlayer and to the substrate by appropriate etching techniques, such as by plasma etching using appropriate gas species for each layer being etched. Depending on the number of layers and materials involved, pattern transfer may involve multiple etching steps using different etching gases. The patterned photoresist layer, photoresist underlayer, and other optional layers in the lithographic stack may be removed following pattern transfer of the substrate using conventional techniques. Optionally, one or more of the layers of the stack may be removed, or is consumed, following pattern transfer to an underlying layer and prior to pattern transfer to the substrate. The substrate is then further processed according to known processes to form an electronic device.

The present inventive concept is further illustrated by the following examples, which are intended to be non-limiting. All compounds and reagents used herein are available commercially except where a procedure is provided below.

### Examples

All reactions were carried out under ambient atmospheric conditions unless stated otherwise. All chemicals were used directly from the supplier. Nuclear magnetic resonance (NMR) spectra for all compounds were obtained on a 400 MHz spectrometer unless otherwise noted. The chemical shifts are reported in 6 (parts per million, ppm) values relative to internal deuterated chloroform residual signal. Multiplicities are indicated by s (singlet), d (doublet), t (triplet), m (multiplet), dd (doublet of doublets), dt (doublet of triplets), tt (triplet of triplets), br (broad singlet).

### Synthesis of N-benzyl-N,N-dimethylbenzenaminium 4-(butoxycarbonyl)-2,6-dihydroxybenzenesulfonate (TAG-1)

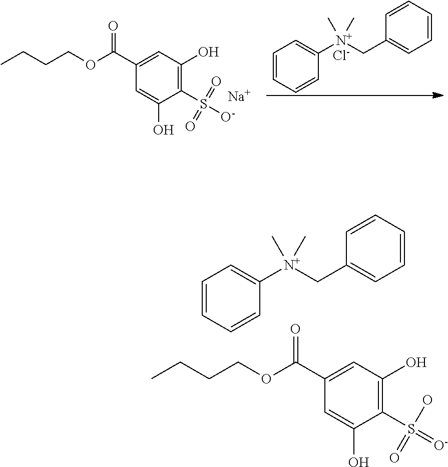

Sodium 4-(butoxycarbonyl)-2,6-dihydroxybenzenesulfonate (10.0 grams (g)) and N-benzyl-N,N-dimethylbenzenaminium chloride (8.73 g) were dissolved in dichloromethane (50 milliliters (mL)) and deionized (DI) water (50 mL) and then stirred at room temperature for 16 hours. The organic layer was washed with DI water (5×25 mL) and concentrated under reduced pressure to afford N-benzyl-N,N-dimethylbenzenaminium 4-(butoxycarbonyl)-2,6-dihydroxybenzenesulfonate (14.1 g, 88%) TAG-1 as a colorless liquid. ¹H NMR (400 MHz, DMSO-d₆) δ 9.98 (s, 2H), 7.85-7.81 (m, 2H), 7.62-7.57 (m, 3H), 7.44-7.39 (m, 1H), 7.33-7.29 (t, 2H), 7.04-7.01 (d, 2H), 6.87 (s, 2H), 5.02 (s, 2H), 4.24-4.21 (t, 2H), 3.59 (s, 6H), 1.70-1.63 (m, 2H), 1.44-1.35 (m, 2H), 0.94-0.90 (t, 3H).

### Synthesis of N-benzyl-N,N-dimethylbenzenaminium 4-(butoxycarbonyl)-2-hydroxybenzenesulfonate (TAG-2)

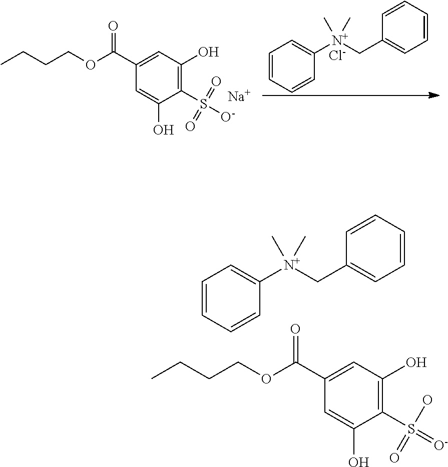

Sodium 4-(butoxycarbonyl)-2-hydroxybenzenesulfonate (7.0 g) and N-benzyl-N,N-dimethylbenzenaminium chloride (6.44 g) were dissolved in dichloromethane (35 mL) and DI water (35 mL) and then stirred at room temperature for 16 hours. The organic layer was washed with DI water (5×17.5 mL) and concentrated under reduced pressure to afford N-benzyl-N,N-dimethylbenzenaminium 4-(butoxycarbonyl)-2-hydroxybenzenesulfonate (10.0 g, 87%) TAG-2 as a colorless liquid. ¹H NMR (400 MHz, DMSO-d₆) δ 10.64 (s, 1H), 7.86-7.81 (m, 2H), 7.63-7.57 (m, 4H), 7.45-7.30 (m, 5H), 7.04-7.01 (d, 2H), 5.01 (s, 2H), 4.27-4.24 (t, 2H), 3.58 (s, 6H), 1.72-1.65 (m, 2H), 1.46-1.37 (m, 2H), 0.95-0.91 (t, 3H).

### Synthesis of 2,4,6-trimethylpyridin-1-ium 4-(butoxycarbonyl)-2,6-dihydroxybenzenesulfonate (TAG-3)

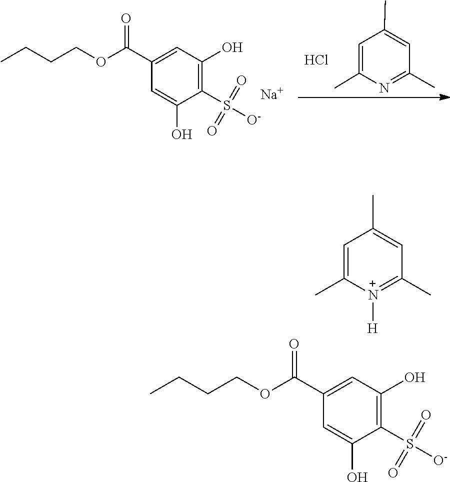

5 normal (N) aqueous hydrochloric acid (3.2 mL) was slowly added to sodium 4-(butoxycarbonyl)-2,6-dihydroxybenzenesulfonate (5.0 g) in water (50 mL) at 0° C. The reaction mixture was stirred for 30 minutes, and then 2,4,6-trimethylpyridine (2.04 g) was added dropwise followed by dichloromethane (50 mL). The biphasic mixture was warmed to room temperature and stirred for 16 hours. The organic layer was separated, washed with water (5×25 mL), precipitated into heptane (500 mL), filtered, and dried to afford 2,4,6-trimethylpyridin-1-ium 4-(butoxycarbonyl)-2,6-dihydroxybenzenesulfonate (4.3 g, 69%) TAG-3 as a white solid. ¹H NMR (400 MHz, DMSO-d₆) δ 14.85 (br, 1H), 9.92 (s, 2H), 7.56 (s, 2H), 6.83 (s, 2H), 4.25-4.21 (t, 2H), 2.62 (s, 6H), 2.49 (s, 3H), 1.71-1.64 (m, 2H), 1.45-1.36 (m, 2H), 0.95-0.91 (t, 3H).

### Synthesis Example 4: Tripropylammonium 4-(butoxycarbonyl)-2,6-dihydroxybenzenesulfonate (TAG-4)

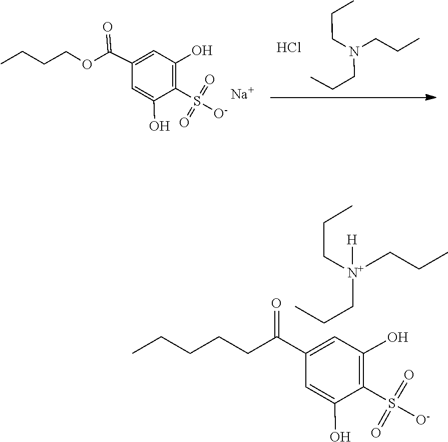

5 N aqueous hydrochloric acid (3.2 mL) was slowly added to sodium 4-(butoxycarbonyl)-2,6-dihydroxybenzenesulfonate (5.0 g) in water (50 mL) at 0° C. The reaction mixture was stirred for 30 minutes, and then tripropylamine (2.41 g) was added dropwise followed by dichloromethane (50 mL). The biphasic mixture was warmed to room temperature and stirred for 16 hours. The organic layer was separated, washed with water (5×25 mL), concentrated under reduced pressure, and dried to afford tripropylammonium 4-(butoxycarbonyl)-2,6-dihydroxybenzenesulfonate (6.1 g, 93%) TAG-4 as a white solid. ¹H NMR (400 MHz, DMSO-d₆) δ 9.92 (s, 2H), 8.86 (br, 1H), 6.84 (s, 2H), 4.25-4.21 (t, 2H), 3.31-2.97 (m, 6H), 1.71-1.57 (m, 8H), 1.45-1.36 (m, 2H), 0.95-0.89 (m, 12H).

### Polymer Synthesis (P1)

Tris (2-hydroxyethyl) iso-cyanurate (15.0 g), tris (2-carboxyethyl) iso-cyanurate (10.0 g), n-butanol (10.1 g), and p-toluenesulfonic acid (0.25 g) were dissolved in anisole (16.7 g). The mixture was heated to 150° C. for 3 hours under a Dean-Stark trap. The mixture was then cooled to room temperature and diluted with methyl 2-hydroxyisobutyrate (80 g). p-Toluenesulfonic acid (0.04 g) and 1,3,4,6-tetrakis(butoxymethyl) tetrahydroimidazo[4,5-d]imidazole-2,5(1H,3H)-dione (8.0 g) were added to a portion of the resulting solution, and heated to 50° C. for 4 hours. The reaction mixture was quenched with triethylamine (0.2 mL), cooled to room temperature, precipitated with isopropanol/heptane, filtered, and dried to afford the desired polymer (12.2 g, 48.8%) P1 as a white powder.

### Preparation of Coating Compositions

Coating compositions were prepared by combining the components in the amounts shown in Table 1. The amounts are expressed in grams. Abbreviations in Table 1 are as follows: A1: tetramethoxymethyl glycoluril; S1: methyl 2-hydroxyisobutyrate; S2: propylene glycol monomethyl ether acetate; S3: propylene glycol methyl ether; TAG-5: N-benzyl-N,N-dimethylbenzenaminium trifluoromethanesulfonate.

TABLE 1

| Coating | | | | |
|---|---|---|---|---|
| Composition | TAG | Polymer | Additive | Solvent |
| 1* | TAG-5 [0.005] | P1 [0.155] | — | S1 [19.84] |
| 2 | TAG 1 [0.003] | P1 [0.157] | — | S1 [19.84] |
| 3 | TAG 2 [0.003] | P1 [0.157] | — | S1 [13.89] |
| | | | | S2 [5.952] |
| 4 | TAG 1 [0.007] | P1 [0.153] | — | S1 [19.84] |
| 5 | TAG 2 [0.007] | P1 [0.153] | — | S1 [13.89] |
| | | | | S2 [5.952] |
| 6 | TAG 3 [0.003] | P1 [0.157] | — | S1 [19.84] |
| 7 | TAG 4 [0.003] | P1 [0.157] | — | S1 [19.84] |
| 8 | TAG 3 [0.003] | P1 [0.133] | A1 [0.024] | S1 [13.89] |
| | | | | S3 [5.952] |
| 9 | TAG 4 [0.003] | P1 [0.133] | A1 [0.024] | S1 [13.89] |
| | | | | S3 [5.952] |

*denotes comparative photoresist composition

### Film Strip Evaluation O

The coating compositions were separately spin coated on a silicon wafer at 1500 rpm using a spinner and the wafer was heated at 205° C. for 60 seconds using a hot plate to form a thin film (film thickness of 200 to 300 Å). The thin film was exposed to 30 mL of PGME/PGMEA (7:3) mixture for 90 seconds and post baked at 110° C. for 60 seconds on a hot plate. Thickness of an initially coated film and an exposed and post baked film were measured, and the change in film thickness (Astrip) was calculated (thickness of an exposed & post baked film—thickness of an initially coated film). The results are shown in Table 3 in angstroms (Å).

| Composition Film | Coating Composition | Δstrip |
|---|---|---|
| Example 1 | 2 | −1.3 |
| Example 2 | 3 | −2.1 |
| Example 3 | 4 | −1.3 |
| Example 4 | 5 | −0.5 |
| Example 5 | 6 | −1.9 |
| Example 6 | 7 | −1.5 |
| Example 7 | 8 | 1.0 |
| Example 8 | 9 | 1.6 |
| Comparative Example 1 | 1 | −26.3 |

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A thermal acid generator compound comprising an anion and a nitrogen-containing cation, wherein the anion is represented by Formula (1): 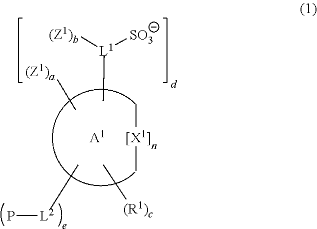
wherein, in Formula (1),
ring A¹ is a monocyclic C₃₋₆₀ aromatic group, a polycyclic C₃₋₆₀ aromatic group, a C₃₋₁₅ monoalicyclic group, or a C₃₋₁₅ polyalicyclic group,
each L¹ is independently a single bond or one or more linking groups, wherein L¹ is free of fluorine,
each L² is independently a single bond or one or more divalent linking groups,
P is a polymerizable group,
each R¹ is independently a monovalent non-hydrogen substituent; wherein each R¹ optionally further comprises one or more divalent linking groups as part of its structure,
each X¹ is independently —O—, —S—, —N(R^{2a})—, —C(O)—, —S(O)R^{2a}—, or —S(O₂)R^{2a}—, wherein R^{2a} is independently chosen from substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl,
each Z¹ independently comprises an anion stabilizing group, wherein at least one Z¹ is configured to form an intramolecular non-covalent bond with the sulfonate anion group to form a ring having from 5 to 9 ring atoms, or from 5 to 8 ring atoms, wherein Z¹ is independently chosen from —OH, —C(O)OH, —SH, —C(O)SH, —NHS(O)₂R³, —S(O)₂R³, —S(O)R³, —S(O)₂NHS(O)₂R³, —CH(═NOH), or —B(R⁴)₂; wherein each Z¹ optionally further comprises one or more divalent linking groups as part of its structure,
each R³ is independently chosen from trifluoromethyl, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl,
each R⁴ is independently chosen from hydrogen, fluorine, hydroxyl, substituted or unsubstituted C₁₋₂₀alkyl, substituted or unsubstituted C₁₋₂₀ heteroalkyl, substituted or unsubstituted C₆₋₃₀ aryl, or substituted or unsubstituted C₃₋₃₀ heteroaryl,
two R¹ together optionally form a fused ring with Cy¹, wherein the fused ring optionally further comprises one or more divalent linking groups as part of its structure,
one Z¹ and one R¹ together optionally form a fused ring with Cy¹, wherein the fused ring optionally further comprises one or more divalent linking groups as part of its structure,
each a and b is independently an integer from 0 to 2, provided that a sum of a and b is 1 or greater,
c is an integer from 0 to 10,
d is an integer from 1 to 3,
e is 0 or 1, and
n is an integer from 0 to 4.

2. The thermal acid generator compound of claim 1, wherein ring A¹ is a monocyclic C₃₋₆₀ aromatic group, or a polycyclic C₆₋₆₀ aromatic group.

3. The thermal acid generator compound of claim 1, wherein ring A¹ is a C₃₋₁₅ monoalicyclic group, or a C₆₋₁₅ polyalicyclic group.

4. The thermal acid generator compound of claim 1, wherein L¹ is a single bond.

5. The thermal acid generator compound of claim 1, wherein the nitrogen-containing cation is ammonium or pyridinium.

6. The thermal acid generator compound of claim 1, wherein at least one anion stabilizing group comprises —OH.

7. The thermal acid generator compound of claim 1, wherein the anion is free of trifluoromethyl groups and difluoromethylene groups.

8. The thermal acid generator compound of claim 1, wherein the anion does not comprise fluorine.

9. The thermal acid generator compound of claim 1, wherein the thermal acid generator compound is polymerizable.

10. A coating composition, comprising:
the thermal acid generator of claim 1;
a polymer; and
a solvent.

11. A patterning method, the method comprising:
applying a layer of the coating composition of claim 10 on a substrate to provide an underlayer composition;
curing the underlayer composition to form an underlayer film;
applying a layer of a photoresist composition on the underlayer film to provide a photoresist composition layer;
pattern-wise exposing the photoresist composition layer to activating radiation to provide an exposed photoresist composition layer; and
developing the exposed photoresist composition layer to provide a resist relief image.

12. The coating composition of claim 10, wherein ring A¹ is a monocyclic C₃₋₆₀ aromatic group, or a polycyclic C₆₋₆₀ aromatic group.

13. The coating composition of claim 10, wherein ring A¹ is a C₃₋₁₅ monoalicyclic group, or a C₆₋₁₅ polyalicyclic group.

14. The coating composition of claim 10, wherein L¹ is a single bond.

15. The coating composition of claim 10, wherein the nitrogen-containing cation is ammonium or pyridinium.

16. The coating composition of claim 10, wherein at least one anion stabilizing group comprises —OH.

17. The coating composition of claim 10, wherein the anion is free of trifluoromethyl groups and difluoromethylene groups.

18. The coating composition of claim 10, wherein the anion does not comprise fluorine.

19. The coating composition of claim 10, wherein the thermal acid generator compound is polymerizable.
